(19) United States
(12) Patent Application Publication
(10) Pub. No.: US 2026/0308688 A1
(43) Pub. Date: Oct. 8, 2026

(54) BIOMARKER FOR PREDICTION OF IMMUNOTHERAPY OUTCOMES AND PRECISION TREATMENT STRATEGIES AND USES THEREOF

(71) Applicant: Ohio State Innovation Foundation, Columbus, OH (US)

(72) Inventor: Jacob KAUFMAN, Columbus, OH (US)

(21) Appl. No.: 19/480,526

(22) PCT Filed: May 3, 2024
(86) PCT No.: PCT/US2024/027739

§ 371 Date: Oct. 31, 2025

Related U.S. Application Data
(60) Provisional application No. 63/463,872, filed on May 3, 2023.

(51) Int. Cl. C12Q 1/6886; C12Q 1/6809

(52) U.S. Cl. CPC C12Q 1/6886; C12Q 1/6809; C12Q 2600/106; C12Q 2600/156; C12Q 2600/158

(57) ABSTRACT

The present disclosure relates to methods of identifying subjects sensitive or resistant to immunotherapy and methods of treating said subjects.

### RELATED APPLICATION

This application claims priority to, and the benefit of, U.S. Provisional Patent Application No. 63/463,872, filed May 3, 2023, which is incorporated by reference herein in its entirety.

### FIELD

The present disclosure relates to biomarkers associated with diagnosing and treating cancers and the uses thereof.

### BACKGROUND

Loss of STK11 occurs in 20-25% of NSCLC and confers poor prognosis and resistance to immunotherapy. While STK11 mutations have been linked to immunotherapy resistance, some STK11-mutant tumors can still respond well to immunotherapy, and loss of STK11 also seems to affect prognosis and response to chemotherapy. The biomarkers and methods of use thereof disclose the need to determine patients' sensitivity to immunotherapy and more.

### SUMMARY

Disclosed herein are methods of predicting responsiveness or resistance of a subject having a cancer to immunotherapeutic agents. The methods and the biomarkers disclosed herein can be used for prediction of immunotherapy outcomes and precision treatment strategies.

In some aspects, disclosed herein is a method of predicting responsiveness of a subject having a cancer to an immunotherapeutic agent, comprising obtaining a biological sample from the subject, determining a level of one or more biomarkers in the biological sample, wherein the one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1, determining an expression score for each of the one or more biomarkers in the biological sample relative to a reference control, determining a mean expression score by averaging the one or more expression scores of the one or more biomarkers, and predicting the subject as responsive to the immunotherapeutic agent if the mean expression score is lower than a threshold.

In some embodiments, the method further comprises predicting the subject as responsive to the immunotherapeutic agent if the mean z-score is lower than 0.5. In some embodiments, the method further comprises administering to the subject a therapeutically effective amount of the immunotherapeutic agent. In some embodiments, the immunotherapeutic agent is an immune checkpoint inhibitor including, but not limited to a PD-1 inhibitor, a PD-L1 inhibitor, a PD-L2 inhibitor, or a CTLA-4 inhibitor. In some embodiments, the checkpoint inhibitor further comprises Pembrolizumab, Nivolumab, Cemiplimab, Atezolizumab, Durvalumab, Ipilimumab, or Tremeliumumab.

In some aspects, disclosed herein is a method of treating a cancer in a subject in need, comprising obtaining a biological sample from the subject, determining a level of one or more biomarkers in the biological sample, wherein the one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1, determining an expression score for each of the one or more biomarkers in the biological sample relative to a reference control, determining a mean expression score by averaging the one or more expression scores of the one or more biomarkers, and administering to the subject a therapeutically effective amount of an immunotherapeutic agent if the mean expression score is lower than a threshold.

In some aspects, disclosed herein is a method of treating a cancer in a subject in need, comprising obtaining a biological sample from the subject, determining a level of a first group of one or more biomarkers, a second group of one or more biomarkers, and a third group of one or more biomarkers in the biological sample, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers comprise one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining an expression score for each of the one or more biomarkers in the biological sample relative to a reference control, determining a first expression score by averaging the expression scores of the one or more biomarkers in the first group, a second expression score by averaging the expression score of the one or more biomarkers in the second group, and a third expression score by averaging the expression score of the one or more biomarkers in the third group, and administering to the subject a therapeutically effective amount of a proinflammatory inhibitor if the first expression score is higher than a first threshold, the second expression score is lower than a second threshold, and the third expression score is lower than a third threshold.

In some embodiments, the method further comprises administering to the subject a therapeutically effective amount of a proinflammatory inhibitor, including but not limited to lenvatinib, sitravatinib, bemcentinib, or an all-trans retinoic acid, if the first z-score is higher than 0.5, the second z-score is lower than 0.5, and the third z-score is lower than 0. In some embodiments, the proinflammatory inhibitor comprises a proinflammatory cytokine inhibitor, including but not limited to an inhibitor of IL-6, IL-15, IL-18, or IL-1.

In some embodiments, the subject has an increased level of one or more inflammatory biomarkers including but not limited to natural killer (NK) cells, CD8+ T cells, polymorphonuclear myeloid-derived suppressor cells (PMN-MDSCs), IL-6, IL-15, IL-18, or IL-1.

In some aspects, disclosed herein is a method of treating cancer in a subject in need, comprising obtaining a biological sample from the subject, determining a level of a first group of one or more biomarkers, a second group of one or more biomarkers, and a third group of one or more biomarkers in the biological sample, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers comprise one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining an expression score for each of the one or more biomarkers in the biological sample relative to a reference control, determining a first expression score by averaging the expression scores of the one or more biomarkers in the first group, a second expression score by averaging the expression score of the one or more biomarkers in the second group, and a third expression score by averaging the expression score of the one or more biomarkers in the third group, and administering to the subject a therapeutically effective amount of a proinflammatory inhibitor if the first expression score is higher than a first threshold, the second expression score is lower than a second threshold, and the third expression score is higher than a third threshold.

In some embodiments, the method further comprises administering to the subject a therapeutically effective amount of an immune checkpoint inhibitor, including but not limited to a PD-1 inhibitor, a PD-L1 inhibitor, a PD-L2 inhibitor, or a CTLA-4 inhibitor, if the first z-score is higher than 0.5, the second z-score is lower than 0.5, and the third z-score is higher than 0. In some embodiments, the checkpoint inhibitor further comprises Pembrolizumab, Nivolumab, Cemiplimab, Atezolizumab, Durvalumab, Ipilimumab, or Tremeliumumab.

In some aspect, disclosed herein is a method of treating a cancer in a subject in need, comprising obtaining a biological sample from the subject, determining a level of a first group of one or more biomarkers and a second group of one or more biomarkers, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1 and the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4, determining an expression score for each of the one or more biomarkers in the biological sample relative to a reference control, determining a first expression score by averaging the expression scores of the one or more biomarkers in the first group and a second expression score by averaging the expression score of the one or more biomarkers in the second group, and administering to the subject a therapeutically effective amount of a TRAF/cIAP inhibitor if the expression score is higher than a first threshold and the second expression score is higher than a second threshold.

In some embodiments, the method further comprises administering to the subject a therapeutically effective amount of a TRAF/cIAP inhibitor, including but not limited to Birinapant, Tolinapant, Xevinapant, or LC-161, if the first mean z-score is higher than 0.5 and the second mean z-score is higher than 0.5.

In some embodiments, the subject has an anti-inflammatory tumor microenvironment. In some embodiments, the subject has a decreased expression level of one or more genes in the TNF/NFKB and/or JAK/STAT signaling pathways. In some embodiments, the subject has an increased expression level of gene TRAF2.

In some aspects, disclosed herein is a method of treating a cancer in a subject in need, comprising obtaining a biological sample from the subject, determining a level of a first group of one or more biomarkers, a second group of one or more biomarkers, and a third group of one or more biomarkers in the biological sample, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers comprise one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining a z-score, or similar relative expression score, for each of the one or more biomarkers in the biological sample relative to a reference control, determining a first z-score by averaging the z-scores of the one or more biomarkers in the first group, a second z-score by averaging the z-scores of the one or more biomarkers in the second group, and a third z-score by averaging the z-scores of the one or more biomarkers in the third group, and administering to the subject a therapeutically effective amount of a proinflammatory inhibitor if the first z-score is higher than 0.5, the second z-score is lower than 0.5, and the third z-score is lower than 0, administering to the subject a therapeutically effective amount of a checkpoint inhibitor if the first z-score is higher than 0.5, the second z-score is lower than 0.5, and the third z-score is higher than 0, or administering to the subject a therapeutically effective amount of an inhibitor of TRAF/cIAP if the first z-score is higher than 0.5 and the second z-score is higher than 0.5.

In some embodiments, the method further comprises determining a level of a fourth group of one or more biomarkers, wherein the fourth group of one or more biomarkers comprise one or more RNAs, or expression products thereof, of the genes selected from the group consisting of HNF4A, MUC13, ANXA13, CDH17, CDX2, AKR1C4, ANKS4B, C12orf27, HNF1A, C10orf108, CDHR2, EPS8L3, TM4SF5, CDHR5, USH1C, HNF4A, SLC39A5, GPR35, MUC13, ANXA10, ANXA13, and CDH17, determining a z-score, or similar relative expression score, for each of the one or more biomarkers in the biological sample relative to a reference control, and determining a fourth z-score by integrating the z-scores of the one or more biomarkers in the fourth group.

In some embodiments, the method further comprises determining a level of a fifth group of one or more biomarkers, wherein the fifth group of one or more biomarkers comprise one or more RNAs, or expression products thereof, of the genes selected from the group consisting of GJB3, GJB5, KRT6A, KRT6B, KRT16, TRIM29, GJB3, GJB4, GJB5, ANXA8L2, ANXA8, KRT6A, KRT6C, KRT6B, KRT16, KRT17, TNS4, PLCD3, and LAMC2, determining a z-score, or similar relative expression score, for each of the one or more biomarkers in the biological sample relative to a reference control, and determining a fifth z-score by integrating the z-scores of the one or more biomarkers in the fifth group.

In some embodiments, the expression score of any preceding aspect is a z-score and the mean expression score is a mean z-score by averaging the one or more z-scores of the one or more biomarkers. In some embodiments, the subject of any preceding aspect has one or more mutations in a STK11 gene. In some embodiments, the subject of any preceding aspect has a normal STK11 gene.

### BRIEF DESCRIPTION OF FIGURES

The accompanying figures, which are incorporated in and constitute a part of this specification, illustrate several aspects described below.

FIGS. 1A, 1B, 1C, 1D, and 1E show a STK11 Functional Loss Score. FIG. 1A shows that the STK11 Functional Loss Score has strong association with STK11 mutations. FIG. 1B shows that this association is true regardless of the type STK11 mutation. FIG. 1C shows receiver operator curve for prediction of STK11 mutations using the STK11 Functional Loss Score, with equivalent results for the ‘mean Z-score’ method and the ‘singscore’ method of score calculation. Data from non-squamous NSCLC from multiple cohorts (N=2118). FIG. 1D shows that in tumor samples where STK11 status is WT or is unknown, that the STK11 Functional Loss Score is strongly associated with loss of STK11 mRNA expression. FIG. 1E shows that in samples that are STK11 WT, the STK11 Functional Loss Score is strongly associated with decreased phosphorylation of AMPK at threonine 172, which is a direct kinase target of STK11. Phosphorylation data are from RPPA analysis of TCGA lung adenocarcinoma samples.

FIGS. 2A, 2B, 2C, and 2D show similar tumor characteristics for NSCLC tumors with STK11 somatic mutations compared to tumors that are STK11 WT that have positive expression of STK11 Functional Loss Score. FIG. 2A shows ‘volcano plot’ depicting statistical association of mRNA differentially expressed genes, comparing STK11 Mutant tumors versus STK11 WT tumors with negative STK11 Functional Loss Score (Score <0.5). FIG. 2B shows ‘volcano plot’ depicting statistical association of mRNA differentially expressed genes, comparing STK11 WT tumors with positive STK11 Functional Loss Score (Score >0.5) versus STK11 WT tumors with negative STK11 Functional Loss Score (Score <0.5). FIG. 2C shows scatter plot of Log Fold Change for mRNA expression with x-axis demonstrating difference between STK11 Mutant tumors versus STK11 WT tumors with negative STK11 Functional Loss Score (Score <0.5) and with y-axis demonstrating difference between STK11 WT tumors with positive STK11 Functional Loss Score (Score >0.5) versus STK11 WT tumors with negative STK11 Functional Loss Score (Score <0.5). FIG. 2D shows the frequency of co-occurring mutations in KEAP1, TP53, KRAS, ATM, SMARCA4, and EGFR for three groups of NSCLC tumors: tumors with somatic mutations in STK11, tumors that are STK11 WT with positive STK11 Functional Loss Score (Score >0.5), and tumors that are STK11 WT with negative STK11 Functional Loss Score (Score <0.5). P-values are calculated using Fisher Exact test and are given for 1) the comparison of combined group of STK11 mutant combined with STK11 WT and positive Functional Loss Score (Score >0.5) versus STK11 WT and positive Functional Loss Score (Score >0.5) which show strong differences between groups, or for 2) STK11 mutant versus STK11 WT and positive Functional Loss Score (Score >0.5), which show no significant differences. Data from non-squamous NSCLC from multiple cohorts (N=2118).

FIGS. 3A and 3B show the association of STK11 Functional Loss Score with immune infiltration score and overall survival of Atezolizumab (anti-PDL1 immunotherapy). FIG. 3A shows that the STK11 Functional Loss Score is associated with decreased immune infiltration score, calculated by Quantiseq algorithm, for NSCLC tumors with STK11 mutations, NSCLC tumors with STK11 WT, and NSCLC tumors with unknown STK11 mutation status. Data from non-squamous NSCLC from multiple cohorts (N=2118). P-value represents student's T-test for comparing STK11 Functional Loss Signature Positive (Score >0.5) vs STK11 Functional Loss Signature Negative (Score <0.5). FIG. 3B shows overall survival of 51 NSCLC patients with STK11 mutations, treated in the Atezolizumab arm (anti-PDL1 immunotherapy) of the OAK clinical trial (NCT02008227). Patients are stratified according to their STK11 Functional Loss Score. P-value and Hazard Ratio are calculated from Cox Proportional Hazard model.

FIGS. 4A, 4B, 4C, 4D, 4E, and 4F show the association of STK11 Loss with distinct differentiation phenotypes. FIG. 4A shows unsupervised UMAP clustering of all non-squamous NSCLC tumors, demonstrating three distinct clusters that are enriched for STK11 mutations. FIG. 4B shows expression of genes comprising the STK11 Functional Loss Gene Set across all non-squamous NSCLC tumors grouped according to UMAP cluster. FIG. 4C shows the STK11 status of NSCLC tumors grouped according to UMAP cluster, indicated as STK11 Mutant, STK11 WT with Positive STK11 Functional Loss Score (Score >0.5), or STK11 WT with Negative STK11 Functional Loss Score (Score <0.5). Cluster 5, 6, and 7 are highly enriched in Functional STK11 Loss and STK11 Mutations. FIG. 4D shows marker genes corresponding to four key MultiLineage DeDifferentiation (MLDD) phenotypes in clusters 5, 6, and 7. These clusters are defined by Neuroendocrine lineage (Cluster 6), Low TTF1 Differentiation (Cluster 5), or Positive TTF1 Differentiation (Cluster 7). Squamous features are variably expressed in tumors with TTF1 Low Differentiation, but rarely expressed in Neuroendocrine or TTF1 Positive groups. Gut-Like features are variably expressed in tumors with TTF1 Low Differentiation as well as tumors with Neuroendocrine differentiation, but are rarely expressed in TTF1 Positive groups. FIG. 4E demonstrates STK11 Functional Loss Score and TTF1 Differentiation Score calculated using malignant cell populations exctracted from single cell RNAseq data. FIG. 4F demonstrates STK11 Functional Loss Score and Neuroendocrine Differentiation Score calculated using malignant cell populations exctracted from single cell RNAseq data. Data in FIGS. 4A, 4B, 4C and 4D are from non-squamous NSCLC from multiple cohorts (N=2118).

FIGS. 5A and 5B show a schema representing the sequential application of three Biomarker Gene Set Scores in a step-wise fashion to yield the STK11-MLDD class for a given non-squamous NSCLC tumor. FIG. 5A shows how this is applied using the ‘Mean Z-score’ method of score calculation. First, the STK11 Functional Loss Signature is calculated to determine a tumor's STK11 functional status. Next, among the STK11 Loss subset, the Neuroendocrine Differentiation Signature is calculated to determine the tumor's neuroendocrine status. Next, among the tumors without neuroendocrine differentiation, the TTF1 Differentiation Signature is calculated to determine the tumor's TTF1 Differentiation Status. Cutoffs shown in FIG. 5A correspond to those used for the ‘Mean Z-score’ method. Cutoffs in FIG. 5B correspond to those used in the ‘singscore’ method.

FIGS. 6A and 6B show differences in tumor phenotypes according to STK11-MLDD class. FIG. 6A shows difference in overall survival for non-squamous tumors from time of resection, grouped according to STK11-MLDD status. Data are from TCGA LUAD and Moffitt (Schabath et al, Oncogene 2016). P-values are from Cox Proportional Hazard Models, comparing each STK11-MLDD class to reference of STK11 WT-Like tumors. FIG. 6B shows mutational differences in KEAP1, KRAS, TP53, EGFR, ATM, and SMARCA4, according to STK11-MLDD class. Two statistical comparisons are shown: 1) STK11 Loss tumors are compared to STK11 WT-Like tumors, regardless of the MLDD status of the STK11-Loss tumors, and 2) Excluding STK11 WT tumors, the mutation frequency is compared across MLDD groups. Data from non-squamous NSCLC from multiple cohorts (N=2118). P-values are from Fisher's Exact Test.

FIGS. 7A, 7B, 7C, and 7D show differences in patient outcomes according to STK11-MLDD status among STK11-deficient tumors treated in the OAK and POPLAR clinical trials. FIG. 7A shows STK11-deficient tumors from the Atezolizumab treatment arm, stratified according to the STK11-MLDD status. STK11-MLDD status significantly impacts overall survival among atezolizumab-treated patients. FIG. 7B shows STK11-deficient tumors from the Docetaxel treatment arm, stratified according to the STK11-MLDD status. STK11-MLDD status has no significant impact for docetaxel-treated patients. FIG. 7C shows STK11-deficient tumors from the Atezolizumab treatment arm, stratified according to the STK11-MLDD status and grouping ‘WT-Like’ and ‘TTF1-Positive’ classes together as an Immune Sensitive group, while grouping ‘Neuroendocrine’ and ‘TTF1-Low’ classes together as Immune Resistant group. FIG. 7D shows STK11-deficient tumors from the Docetaxel treatment arm, stratified according to the STK11-MLDD status and grouping ‘WT-Like’ and ‘TTF1-Positive’ classes together as an Immune Sensitive group, while grouping ‘Neuroendocrine’ and ‘TTF1-Low’ classes together as Immune Resistant group. Data are from OAK (NCT02008227) and POPLAR (NCT01903993) clinical trials. STK11-deficient tumors depicted here either have presence of STK11 mutation or are STK11-WT or STK11-unknown with STK11 Functional Loss score >0.5. P-value and Hazard Ratio are calculated from Cox Proportional Hazard model.

FIGS. 8A, 8B, and 8C show differences in patient outcomes according to STK11-MLDD status among STK11-deficient tumors treated with Atezolizumab in the OAK and POPLAR clinical trials, among specific patient subgroups of special importance due to previously reported associations with immunotherapy outcomes. FIG. 8A shows difference in overall survival among STK11-deficient tumors that are KEAP1 WT, according to STK11-MLDD status. FIG. 8B shows difference in overall survival among STK11-deficient tumors that are KEAP1 Mutant, according to STK11-MLDD status. FIG. 8C shows difference in overall survival among STK11-deficient tumors that have PDL1 expression <1%, according to STK11-MLDD status. STK11-deficient tumors depicted here either have presence of STK11 mutation or are STK11-WT or STK11-unknown with STK11 Functional Loss score >0.5. MLDD-Sensitive refers to the tumors with ‘WT-Like’ or ‘TTF1-Positive’ MLDD class. MLDD-Resistant refers to the tumors with ‘Neuroendocrine’ or ‘TTF1-Low’ MLDD Class. Data are from OAK (NCT02008227) and POPLAR (NCT01903993) clinical trials. P-value and Hazard Ratio are calculated from Cox Proportional Hazard model.

FIGS. 9A, 9B, 9C, 9D, and 9E show evaluation of key immune signaling pathways according to STK11-MLDD status, demonstrating lowest activation in STK11-Neuroendocrine class. FIG. 9A shows results from Hallmark gene set analysis for ‘TNF Signaling via NFKB’, ‘Interferon Gamma Response’, ‘IL6 JAK STAT3’, and ‘Apoptosis’ Hallmark pathway gene sets. Gene sets are taken from molecular signatures database (msigDB), and numeric score is calculated by taking the mean of genes comprising each gene set shown. FIG. 9B shows representative marker genes from Major Histocompatibility Complex 1 pathway: HLA-A, HLA-B, TAPI, PSMB8. FIG. 9C shows representative marker genes from Interferon Gamma Response pathway: IFIT1, IFI44, OAS2, MX1. FIG. 9D shows marker genes from Apoptosis pathway CASP1, CASP4, FAS, TNFSF10 (TRAIL). FIG. 9E shows marker genes from the CGAS, STING, IFI16 pathway. Data are from non-squamous NSCLC from multiple cohorts (N=2118). P-values represent results of Student's T-test comparing indicated patient groups.

FIGS. 10A, 10B, 10C, 10D, and 10E show evaluation of the immune composition of the tumor microenvironment according to tumor STK11 and MLDD status, with decreased immune infiltration in STK11 deficient tumors, and specific alterations of immune composition distinguishing STK11 deficient tumors from STK11 WT tumors. FIG. 10A shows estimate of total immune infiltration calculated using xCell algorithm (RStudio) with tumors grouped by STK11-MLDD class. FIG. 10B shows estimate of total immune infiltration calculated using Timer algorithm (RStudio) with tumors grouped by STK11-MLDD class. FIG. 10C shows estimate of total immune infiltration calculated using Quantiseq algorithm (RStudio) with tumors grouped by STK11-MLDD class. The y-axis in FIGS. 10A, 10B, and 10C represents the z-score of the respective estimate of total immune infiltration. FIG. 10D represents results of immune deconvolution using the xCell algorithm, comparing the relative composition for each cell type between STK11 deficient tumors and STK11 WT tumors, classified according to the STK11 Functional Loss Score. FIG. 10E represents results of immune deconvolution using the Cibersort algorithm, using a customized cell reference matrix based on NSCLC single cell RNA sequencing data, and comparing the relative composition for each cell type between STK11 deficient tumors and STK11 WT tumors, classified according to the STK11 Functional Loss Score. Both FIG. 9D and FIG. 9E demonstrate associations of increased plasma cell infiltration, decreased dendritic cell infiltration, and increased neutrophil infiltration. Data from non-squamous NSCLC from multiple cohorts (N=2118). P-values in FIGS. 10D and 10E reflect results of Student's T-test comparing STK11-Loss and STK11 WT-Like tumors. Red color indicates increased levels in STK11-Loss tumors; blue color indicates decreased levels in STK11-Loss tumors.

FIGS. 11A, 11B, 11C, and 11D show evaluation of neutrophil and myeloid derived suppressor cell (MDSC) phenotypes according to STK11-MLDD class. FIG. 11A represents the composition of neutrophils in the tumor microenvironment, relative to the total immune infiltrate. Immune deconvolution estimates were made using the Cibersort algorithm using a custom reference cell matrix generated from single cell RNA sequencing data from human non-small cell lung cancer to ensure the greatest applicability to this dataset. FIG. 11B shows marker genes CXCR1, ADGRG3, ILIR2, and FCGR3B visualized to be highly neutrophil specific using single cell RNA sequencing data of NSCLC (Maynard et al, Cell 2020). Neutrophil cell cluster is indicated with red arrow. FIG. 11C shows expression levels of CXCR1, ADGRG3, ILIR2, and FCGR3B across NSCLC patients grouped by STK11-MLDD class. FIG. 11D shows expression levels of marker genes known to be markers of myeloid derived suppressor cells (MDSCs) across NSCLC patients grouped by STK11-MLDD class. Data in FIGS. 11A, 11C, and 11D from non-squamous NSCLC from multiple cohorts (N=2118). P-values represent results of Student's T-test comparing indicated patient groups.

FIGS. 12A, 12B, 12C, 12D, 12E, and 12F show evaluation of dendritic cell populations and their association to STK11-MLDD class. FIG. 12A shows the abundance of conventional dendritic cell 2 (cDC2), calculated using Cibersort immune deconvolution algorithm, relative to total immune infiltration for NSCLC tumors according to STK11-MLDD class. FIG. 12B shows associated dendritic cell marker genes according to STK11-MLDD class. FIG. 12C shows expression of gene signature representing Major Histocompatibility Complex 2 (MHC2) for NSCLC tumors according to STK11-MLDD class. FIG. 12D shows associated MHC2 marker genes for NSCLC tumors according to STK11-MLDD class. FIG. 12E shows cDC2 abundance as measured from single cell RNAseq analysis of NSCLC tumors. Individual cells annotated as ‘Dendritic cell cDC2’ were quantified for each tumor sample, and divided by the total number of annotated immune cells to give a relative abundance. The cDC2 abundance is plotted versus the TTF1 Differentiation score and the STK11-MLDD is colored as shown. FIG. 12F shows the relative cDC2 abundance according to STK11-MLDD class. Data in FIGS. 12A, 12B, 12C, 12D are bulk expression data from non-squamous NSCLC from multiple cohorts (N=2118). Data in FIGS. 12E and 12F are single cell RNAseq expression data (N=157, Salcher et al. Cancer Cell 2022). P-values represent results of Student's T-test comparing indicated patient groups.

FIGS. 13A, 13B, 13C, and 13D show that the TTF1 Differentiation status of a tumor alters is associated with increased expression of a key myeloid derived suppressor cell gene, S100A9, and decreased expression of MHC-2 antigen presentation genes within myeloid cells. FIG. 13A demonstrates that S10049 is a highly over-expressed gene in multiple myeloid cell types when comparing cells from TTF1 Positive tumors vs TTF1 Low tumors. This was a top differentially expressed gene for monocytes, neutrophils, macrophages, and cDC2 dendritic cells. FIG. 13B shows the inverse relationship between S100A9 and CD74 within monocytes. CD74 is a representative MHC-2 gene important for antigen presentation. Monocytes within TTF1 Low tumors exhibit a shift towards S100A9 high, CD74 low expression, while monocytes in TTF1 High tumors express higher fraction of CD74 high, S10049 low cells. FIG. 13C shows the inverse relationship between S100A9 and CD74 within macrophages. Macrophages within TTF1 Low tumors exhibit a shift towards S100A9 high, CD74 low expression, while macrophages in TTF1 High tumors express higher fraction of CD74 high, S10049 low cells. FIG. 13D shows the inverse relationship between S100A9 and either CD74 or HLA-DRA—two MHC-2 genes—in multiple myeloid populations: cDC2, monocytes, and macrophages, concluding that increased expression of S10049 in a tumor sample is highly correlated with decreased MHC-2 antigen presentation within myeloid cells. In FIGS. 13B and 13C each point represents a single cell annotated as being monocyte (in 13B) or macrophage (in 13C). In FIG. 13D each point represents a single tumor. P-values in FIG. 13D result from general linear regression model, as shown. Data are from single cell RNAseq analysis of non-squamous NSCLC from Salcher et al, Cancer Cell 2022; N=157 tumors.

FIGS. 14A, 14B, 14C, 14D, and 14E show results from analysis of murine tumors from GEMM model of STK11 mutation. FIG. 14A is a schema depicting the specific GEMM cell lines used, the genotype of each cell line, number of mice injected, and analytical approach which performed gene expression analysis using a custom NanoString nCounter platform (200 genes) to evaluate STK11-MLDD phenotypes, immune signaling pathways, and features of the immune microenvironment, and further analyzed tumor immune biology using a 33 color spectral flow cytometry analysis with CD8 T-cell antibody panel. Cell line 2381T4S has KRAS-G12D, STK11 null, and TP53 null genotype, exhibits TTF1-Low MLDD class with squamous features. Cell line 2695X has KRAS-G12D, STK11 null, and TP53 null genotype, exhibits TTF1-Low MLDD class with gut-like features. Cell line 2695WT is an isogenic derivative of 2695X with re-expression of STK11 WT. Four cell lines representing KRAS-G12D and TP53 null genotype are used as STK11 WT controls. FIG. 14B shows a heatmap representing gene expression from NanoString panel integrated with CD8 T-cell phenotypes from spectral flow cytometry. Neutrophil/MDSC markers, MHC-2, IFNG, and flow cytometry phenotypes corresponding to Naïve CD8 T-cells, Effector CD8 T-cells, and Exhausted CD8 T-cells are shown. FIG. 14C shows box plots of gene expression MDSC markers S100a8, S100a9, and MHC-2 genes Cd74, and H2-dma, across the four cell line classes. FIG. 14D shows scatter plots of gene expression for MDSC and neutrophil marker genes S100a8, S100a9, Cxcr2, and Csf3r. These markers show strong positive correlation, consistent with detection of increased MDSC-Neutrophils. FIG. 14E shows scatter plots of gene expression for MDSC marker genes S100a9, and MHC-2 genes Cd74, and H2-dma. The MHC-2 genes show strong inverse correlation with S100a9, with the same relationship observed in human single cell RNAseq data.

FIGS. 15A, 15B, 15C, and 15D show results from spectral flow cytometry characterization of CD8 T-cells from murine experiment. FIG. 15A shows UMAP analysis of CD8 T-cells analyzed with 33 color spectral flow cytometry. Key functional clusters are annotated for Naïve, early effector, and terminally exhausted phenotypes. FIG. 15B shows density plots of same UMAP analysis for tumors from STK11 Mutant 2695X cell line, compared to isogenic STK11 WT derivative, 2695WT. This shows increase in effector cells in STK11 WT tumors, and decreased terminally exhausted CD8 cells in STK11 mutant tumors, with TOX expression shown for reference. FIG. 15C quantitates the relative abundance of each CD8 UMAP cluster for STK11 Mutant vs STK11 WT derivatives of the 2695X cell line. FIG. 15D demonstrates inverse relationship between S100A9 MDSC marker and total CD8 infiltration, as well as positive correlation with Naïve T-cell cluster, for 2695X, 2695WT, and 2381T4X cells.

FIGS. 16A, 16B, 16C, 16D, and 16E show associations of STK11 Functional Loss Signature applied to human NSCLC cell lines, and association with potential therapeutic targets. FIG. 16A shows the STK11 Functional Loss Signature expression among human NSCLC cell lines from CCLE. STK11 mutant cell lines exhibit clear increase in expression of STK11 functional loss score. However, a subset of cell lines reported to have STK11 mutation exhibit lower STK11 Functional Loss Score at levels consistent with STK11 WT-Like biology. FIG. 16B demonstrates how this variability in STK11 Functional Loss score can be leveraged to identify genes and pathways that are essential to the growth of STK11 deficient cell lines, using CRISPR dependency data from the DepMap database. Genes with negative ‘essentiality scores’ have significant inhibitory growth effect when targeted by CRISPR guide RNAs in these screens. Highlighted dependency data include NFE2L2, with known strong links to STK11 biology, novel associations with other transcription factors HNF1B, FOXK1, FOXA1, and KLF5, and signaling pathway genes IRS2, IGF1R, and CCND3. FIG. 16C demonstrates a novel pathway dependency with high phenotypic effect of insulin like growth factor receptor (IGF1R) and insulin receptor substrate 2 (IRS2). These dependencies are closely correlated with each other. Evaluation of an IGF1R pathway activation signature comprising IRS2, IGF1R, FURIN, and INSL4 showed very strong association with IRS2 CRISPR essentiality score. FIG. 16D shows association between STK11 Functional Loss Signature and susceptibility to small molecule inhibitors of cIAP1-birinapant, and LCL-161. FIG. 16E shows association between STK11 Functional Loss Signature and susceptibility to inhibitors of cyclin dependent kinases—Palbociclib, NU6027, arcyriaflavin.a, and CRISPR dependency for CCND3. Association with CRISPR phenotypes in FIG. 16B, 16C, and 16E represent the result of linear regression model versus STK11 Functional Loss Signature Links to CRISPR essentiality score from CCLE DepMap data. Association with small molecule inhibitors in FIG. 16D and FIG. 16E were identified by interrogating the CTRP and GDSC databases of in vitro cell line drug susceptibility for significant associations using general linear model versus STK11 Functional Loss Signature. P-values represent results from general linear model.

FIGS. 17A, 17B, 17C, 17D, 17E, and 17F show association between STK11-MLDD phenotypes and TRAF2/cIAP1, suggesting that this important anti-apoptotic and immune resistance pathway may represent a therapeutic target for STK11-deficient. FIG. 17A shows that TRAF2 is highly upregulated on the mRNA and protein level in NSCLC with STK11 loss as determined by STK11 functional loss signature. Expression of other TRAF and BIRC family members is evaluated and shows no significant increased association. FIG. 17B shows that protein levels of TRAF2 and its binding partner cIAP1 are highly correlated. Both proteins are over-expressed in STK11 Deficient NSCLC. Of note, mRNA levels for cIAP1 (gene name BIRC2) do not show these correlations, strongly implicating stabilization of cIAP1 due to TRAF2 over-expression. FIG. 17C shows mRNA expression of TRAF2 according to STK11-MLDD status, with highest levels detected in STK11 Loss Neuroendocrine tumors. FIG. 17D shows inverse association of TRAF2 mRNA with immune infiltration across STK11-MLDD groups, with strong inverse association seen particularly for STK11 Neuroendocrine tumors. FIG. 17E shows inverse association of TRAF2 mRNA with NFKB signature across STK11-MLDD groups, with strong inverse association seen particularly for STK11 Loss Neuroendocrine tumors and STK11 Loss TTF1 Positive tumors, inverse association of lower magnitude for STK11 Loss TTF1 Low, and minimal effect in STK11 WT. FIG. 17F shows inverse associations of TRAF2 with individual immune signaling and response genes of interest, comparing NSCLC with expression of STK11 Functional Loss Score vs STK11 WT-Like. Proteomic data in 17A and 17B are from CPTAC lung adenocarcinoma study (N=110, Gillette et al, Cancer Cell 2020). Genomic data in 17A, 17C, 17D, 17E, and 17F are bulk expression data from non-squamous NSCLC from multiple cohorts (N=2118). P-values in 17A and 17C represent results of Student's T-test comparing indicated groups. P-values and regression estimates in 17B, 17D, 17E, and 17F represent results of general linear models.

FIGS. 18A, 18B, and 18C represent in vitro assessment of tumor cell susceptibility to CD8 cytotoxicity, and synergistic effects of cIAP1 inhibition for STK11 mutant cell lines. FIG. 18A shows effects on cell viability of co-culture of target tumor cell lines expressing ovalbumin, either STK11 mutant 2695X murine cell line or its STK11 WT isogenic derivative 2695WT, co-cultured with OT-I CD8 T-cells at three T-cell to Tumor cell ratios. STK11 WT derivative shows increased susceptibility to CD8 cytotoxicity. FIG. 18B demonstrates that treatment with cIAP1 inhibitor birinapant at 500 nM significantly enhances cytotoxic effect in STK11 mutant cell line, without appreciable added effect for STK11 WT derivative. Cytotoxic effects are reversed with use of RIPK3 inhibitor. FIG. 18C shows Bliss index calculations evaluating for synergy between cIAP1 inhibitor birinapant and OT-I co-culture. Significant synergy is observed in STK11 mutant setting, with no appreciable synergy detected for STK11 WT derivative. These results further suggest that TRAF2/cIAP1 may represent a precision therapeutic target to enhance immune response in the setting of STK11 loss.

FIG. 19 shows a schema summarizing the main differences in signaling and immune microenvironment between STK11 Loss Neuroendocrine and STK11 Loss TTF1 Low, which are the two STK11-MLDD classes that appear to be most immune resistant. STK11 Loss Neuroendocrine tumors have significant down regulation of most immune signaling pathways including NFKB, Interferon Gamma Response, IL6-JAK-STAT3, and MHC-1 expression. They have the highest levels of TRAF2/cIAP1, which appears to be strongly anticorrelated with each of these pathways, suggesting possible functional antagonism. Conversely, TTF1 Low tumors express the highest levels of inflammatory cytokines IL6, IL15, and IL18, and appear to have highest levels of neutrophils and specifically MDSC phenotypes which we link to S100A8/S100A9 expression as putative marker gene. MDSCs have been shown to be functionally immunosuppressive in STK11 deficient NSCLC in multiple prior studies. Therapeutic strategies targeting MDSC biology or disrupting S100A8/S100A9 immune suppressive activity may have utility in TTF1 Low tumors. S100A8/S100A9 expression correlates with low dendritic cell recruitment and low MHC2 expression, and may be associated with naïve T-cell phenotype in our in vivo studies. Low dendritic cell recruitment, low MHC2 expression, and low STING expression are seen in both neuroendocrine and TTF1-Low subsets, with STK11 Loss TTF1 positive subsets having levels much closer to those seen in STK11 WT tumors, which may in part explain their apparent immune responsiveness in our OAK and POPLAR analyses.

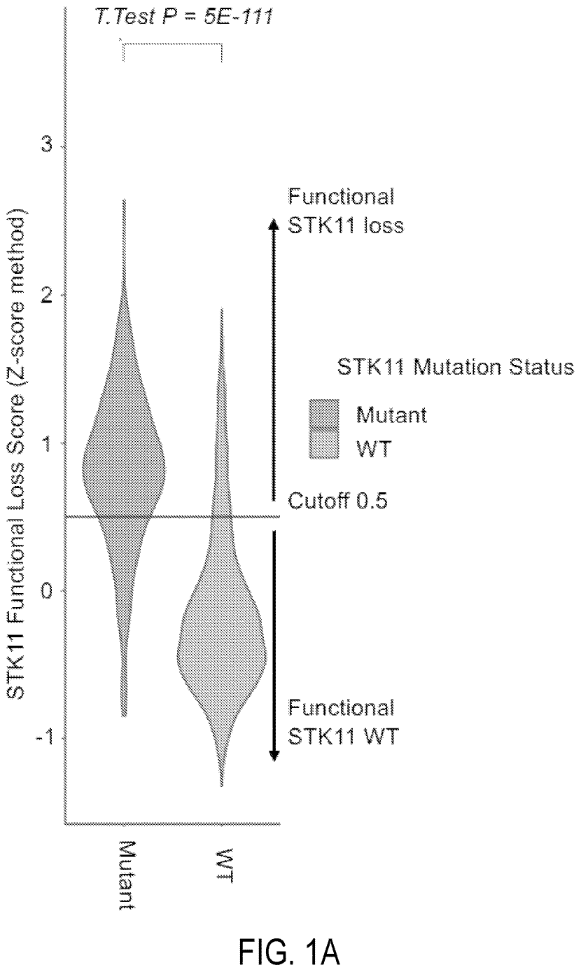

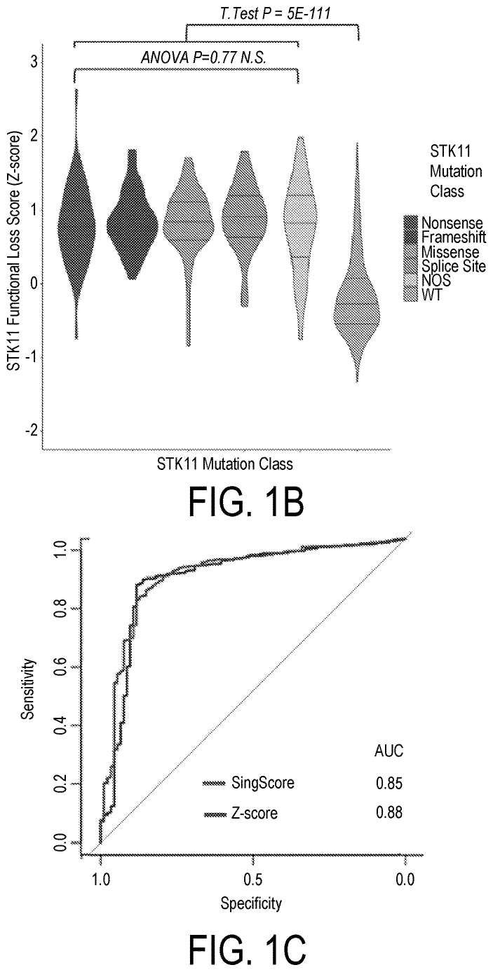

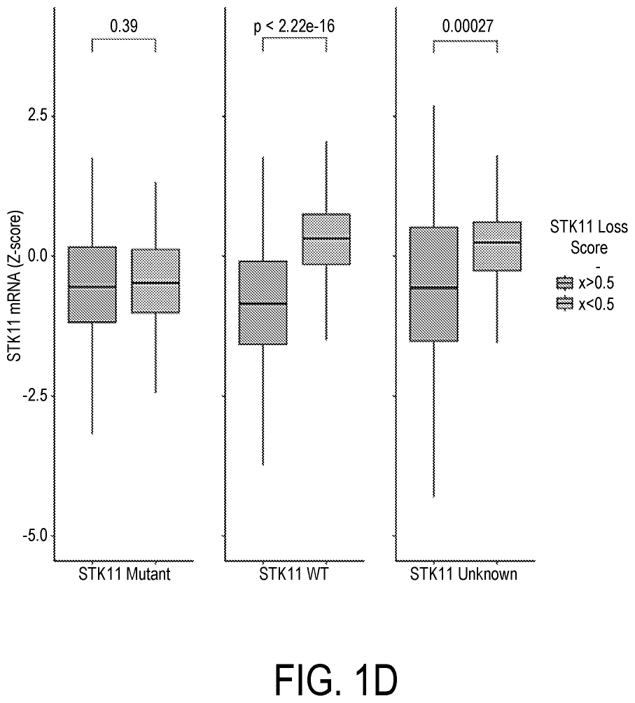

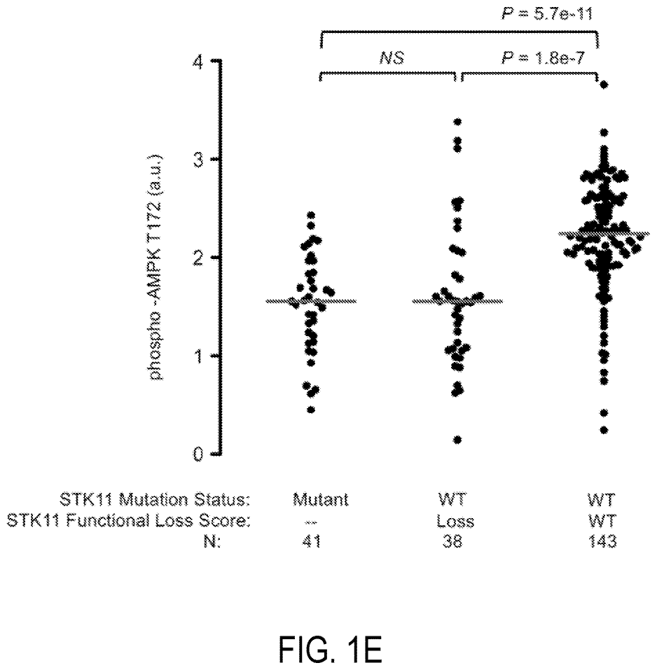

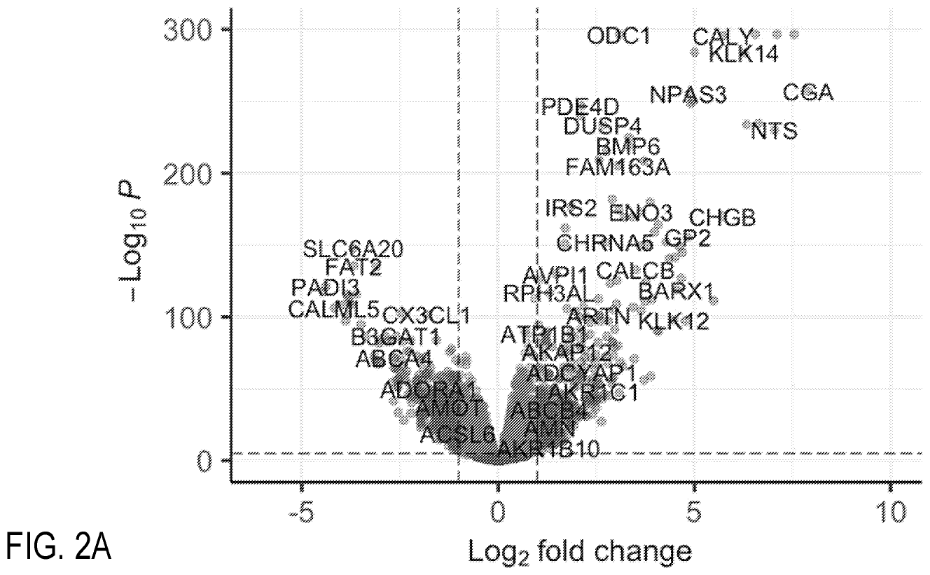

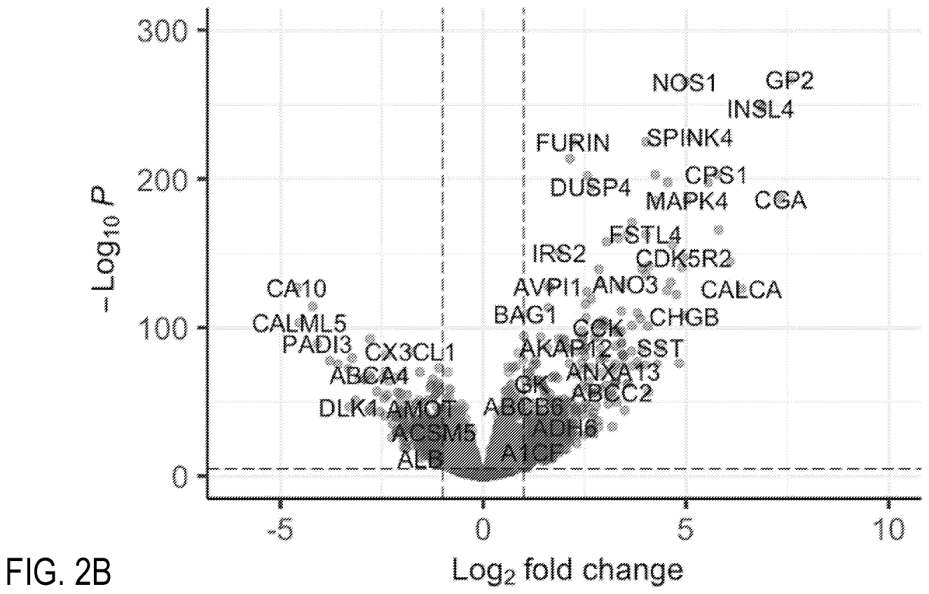

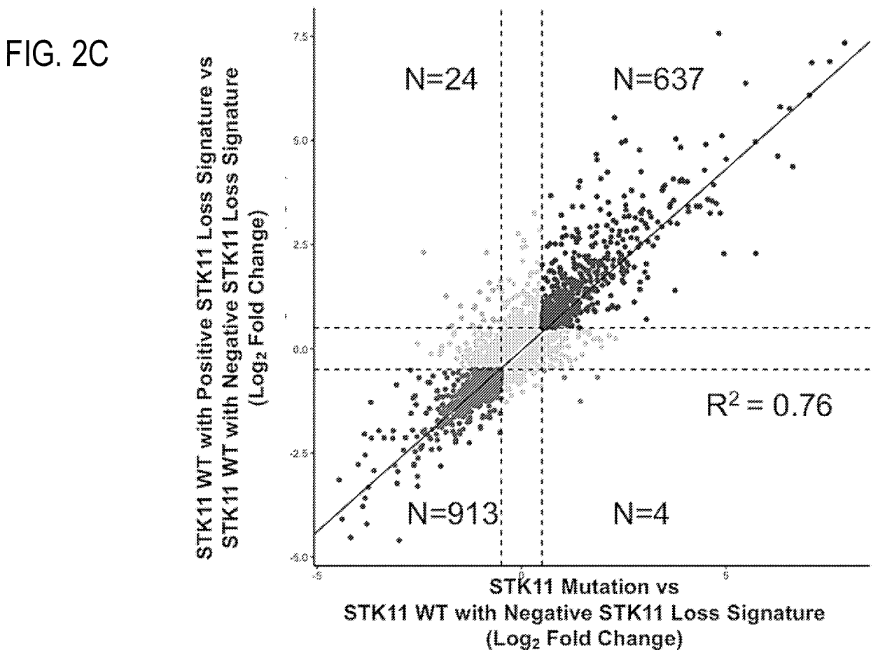

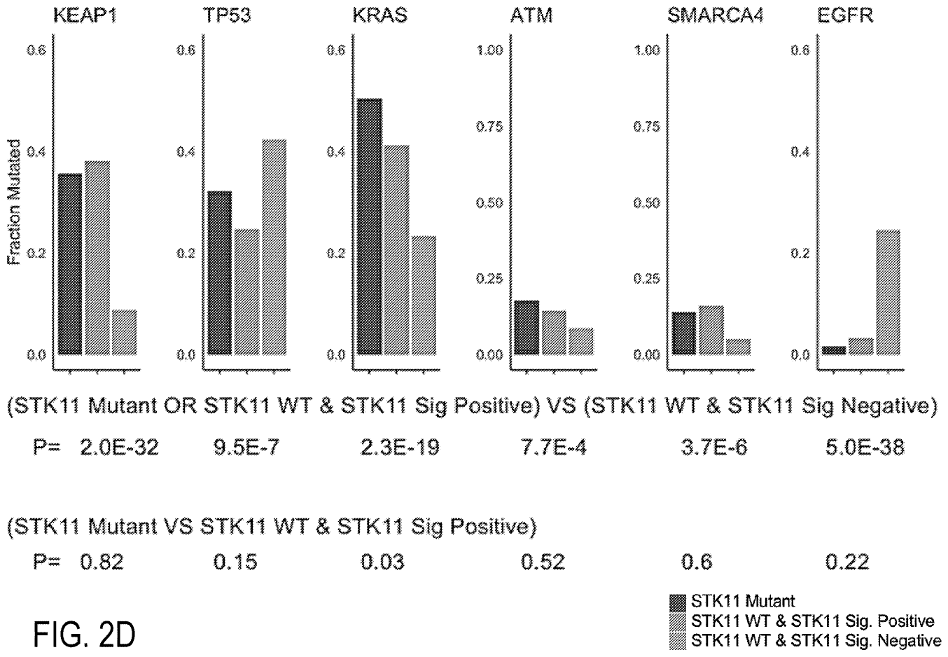

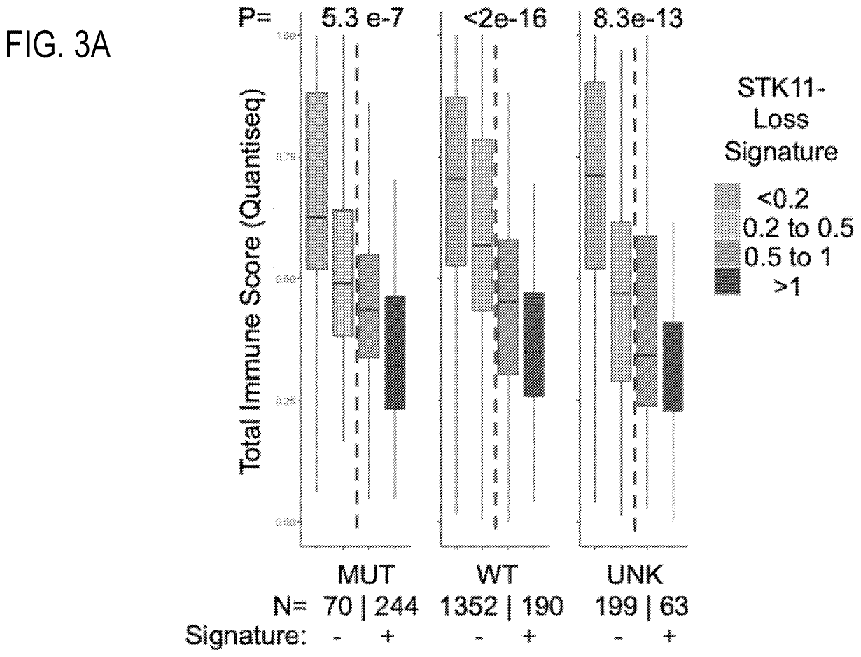

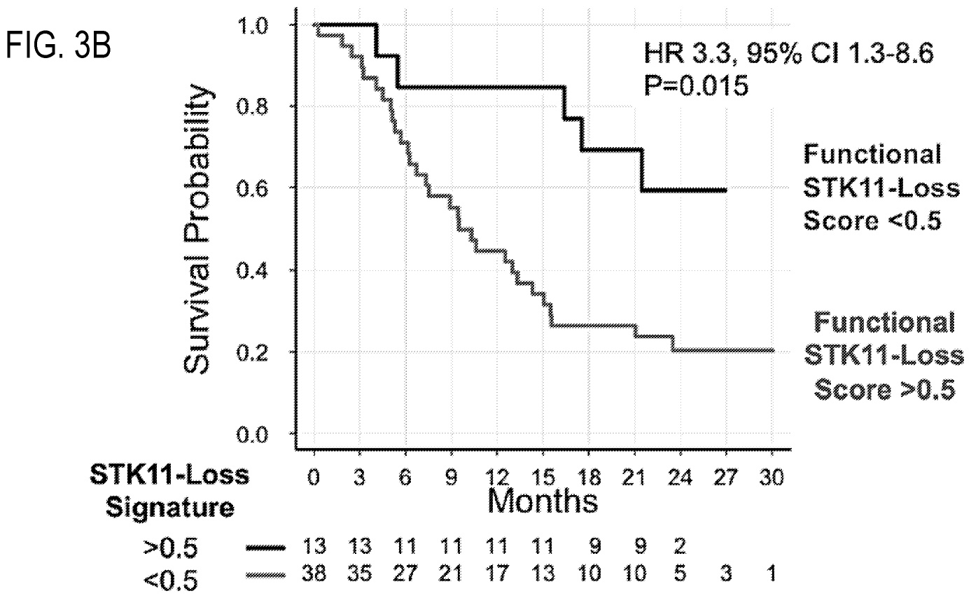

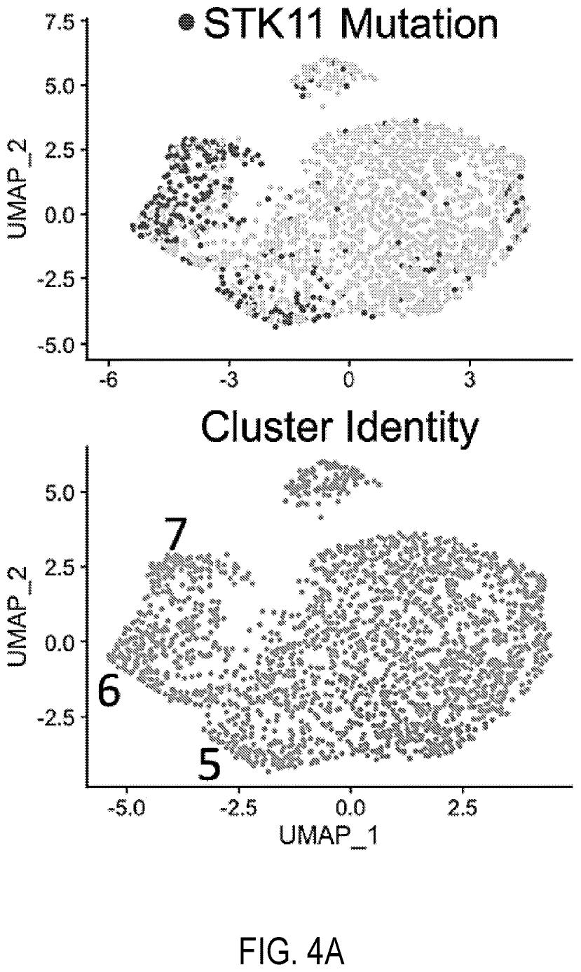

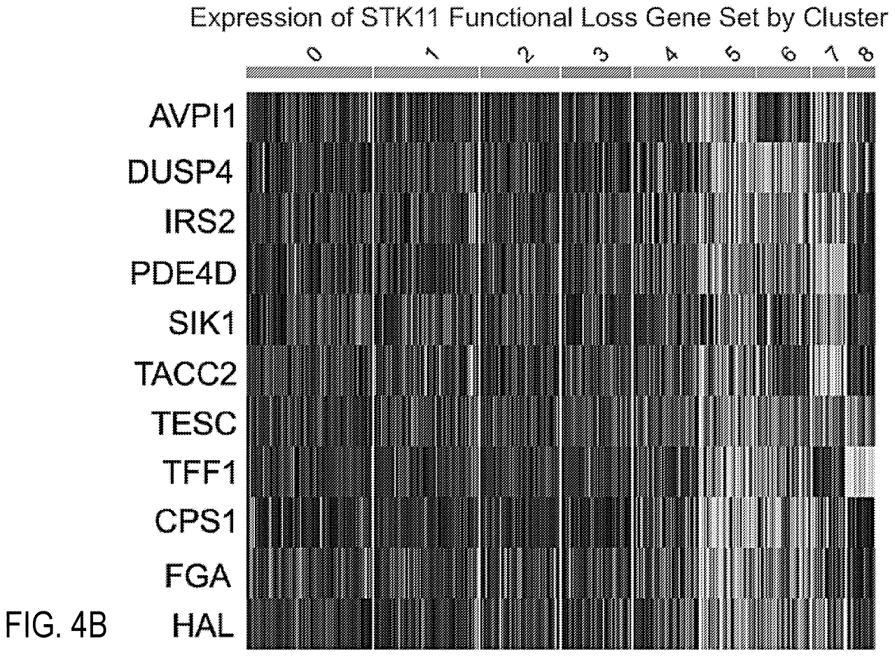

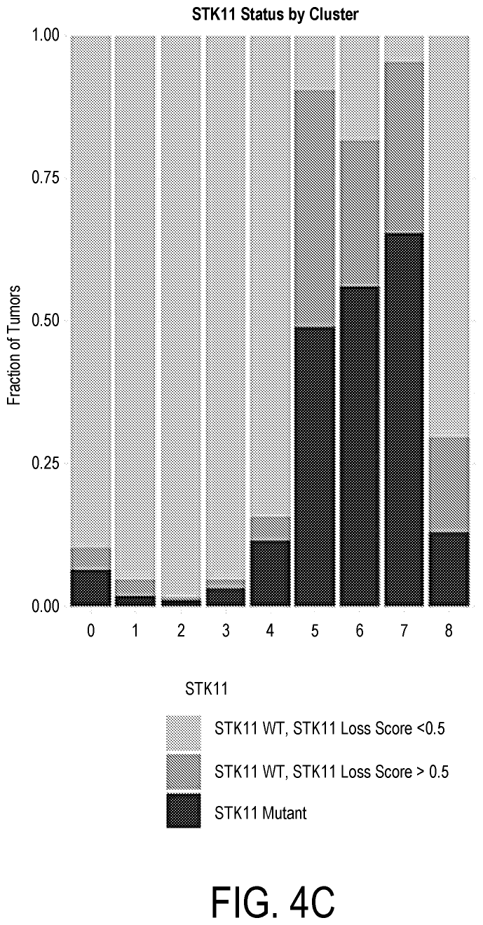

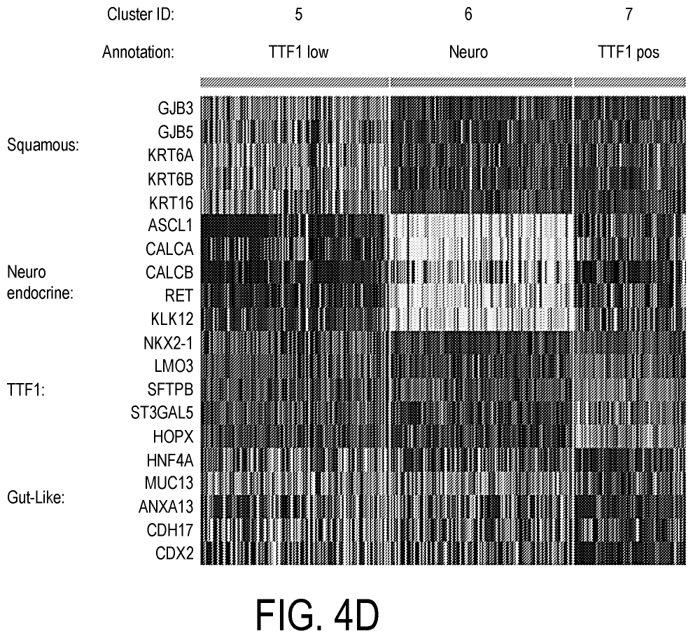

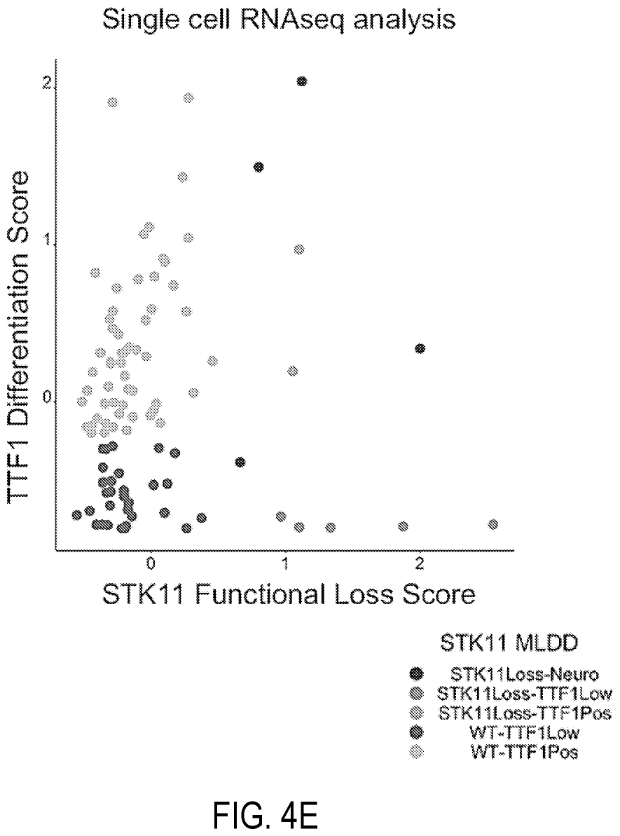

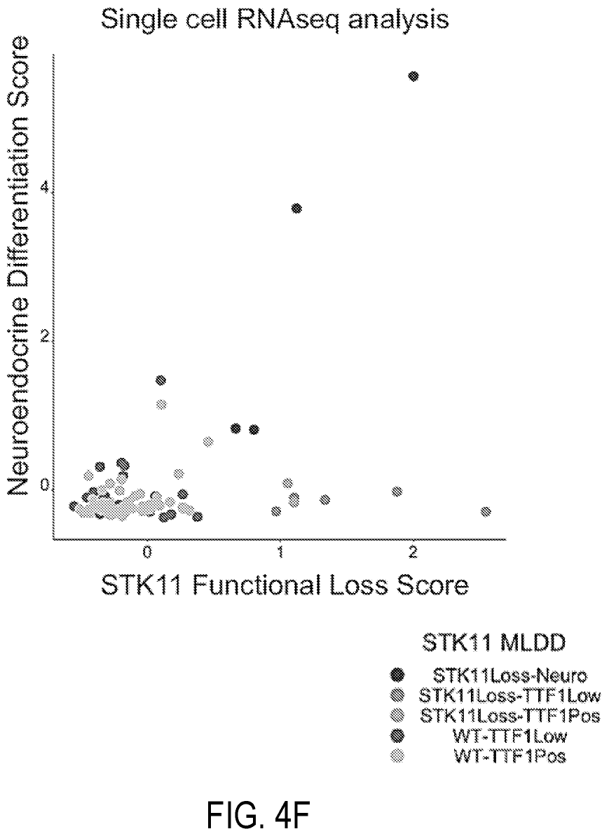

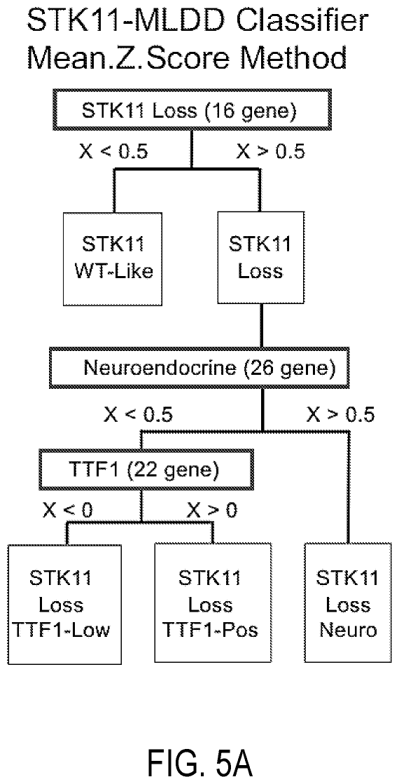

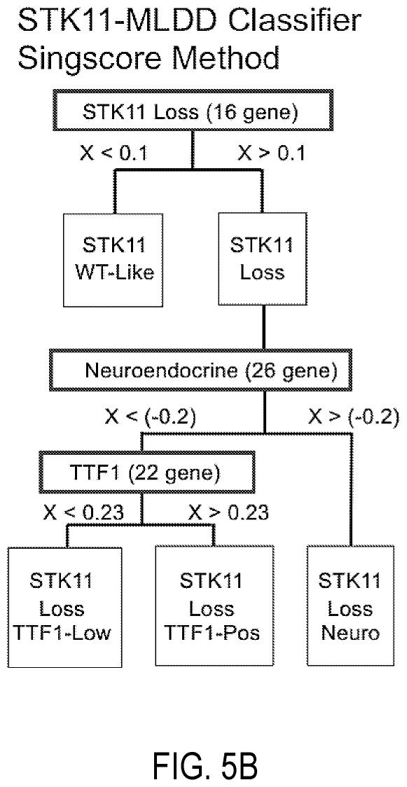

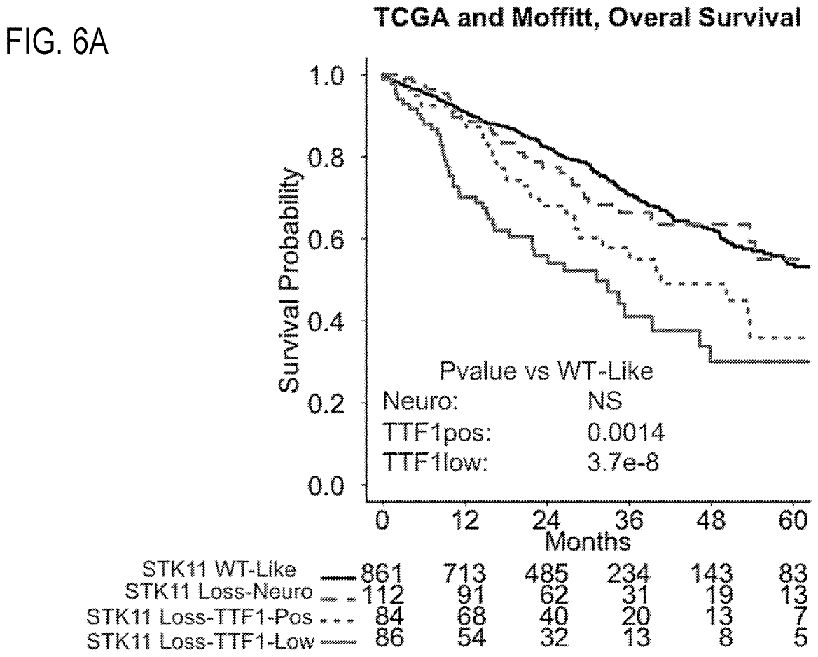

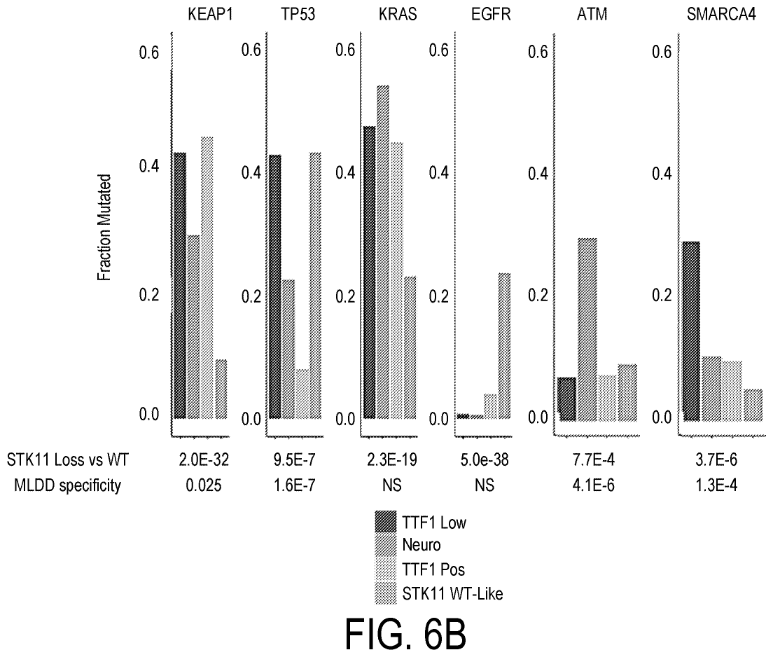

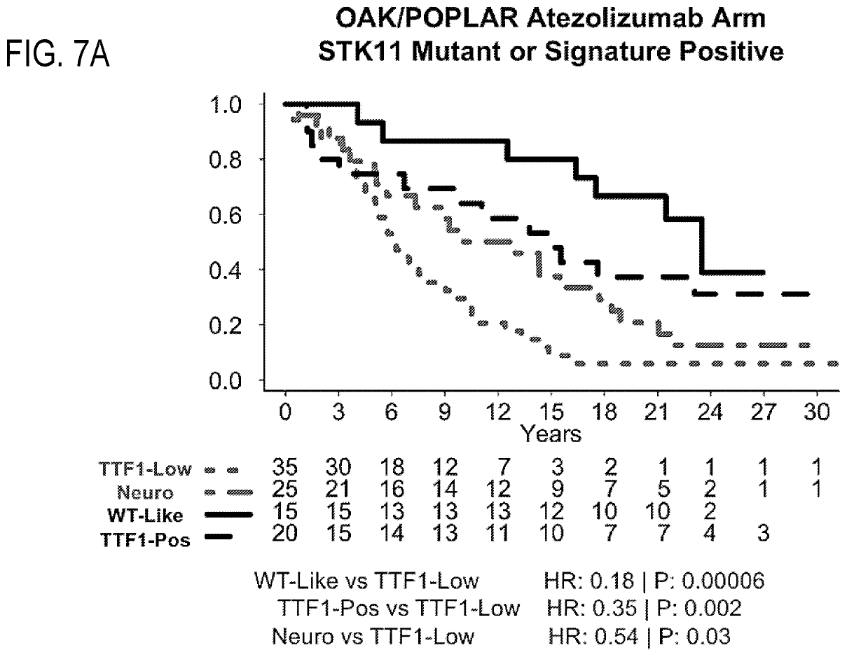

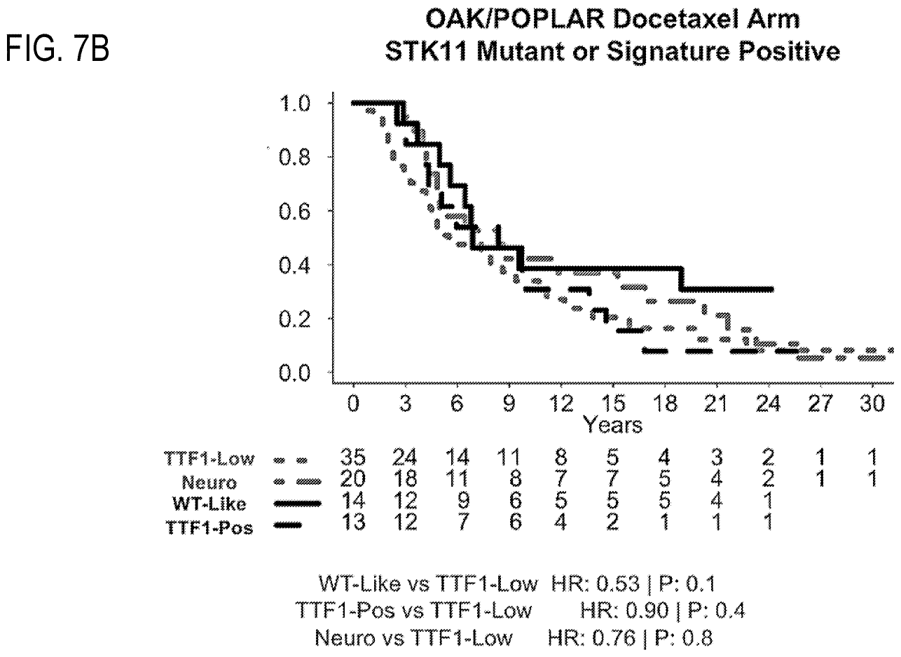

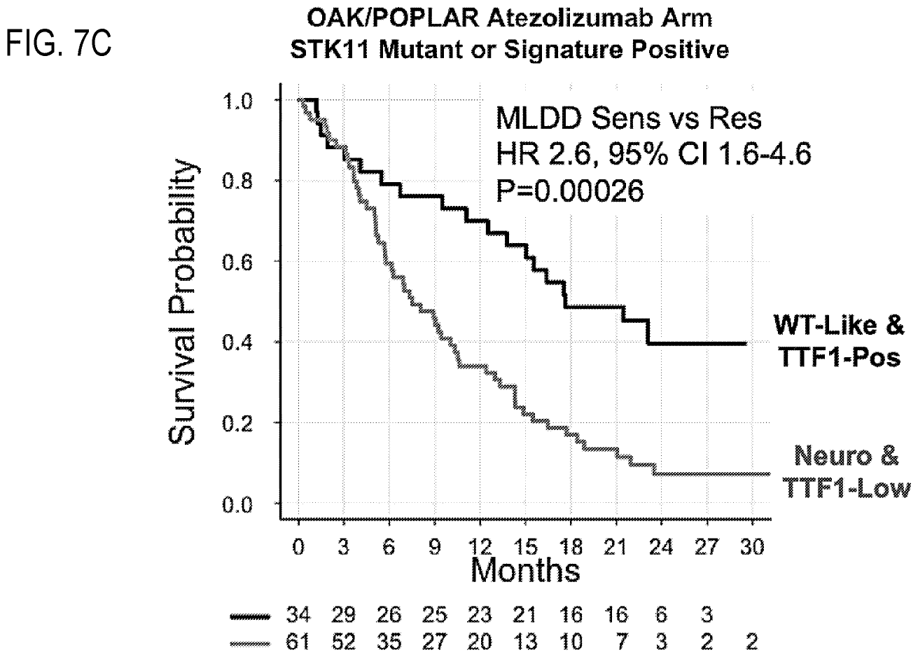

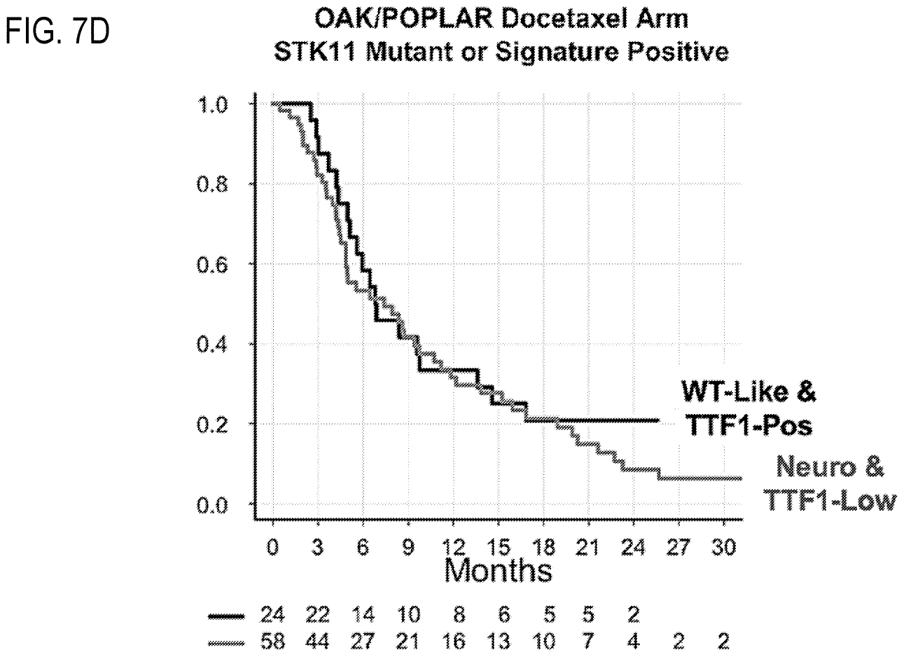

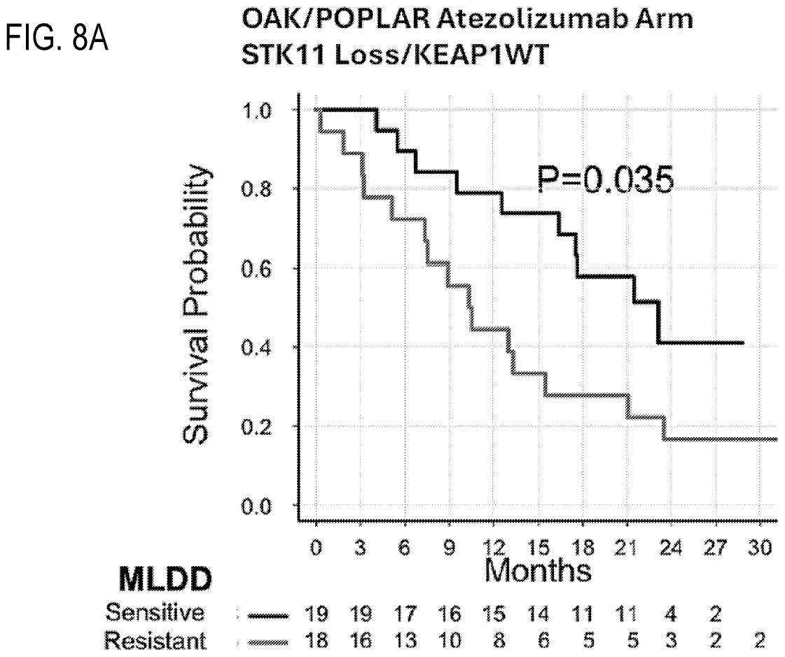

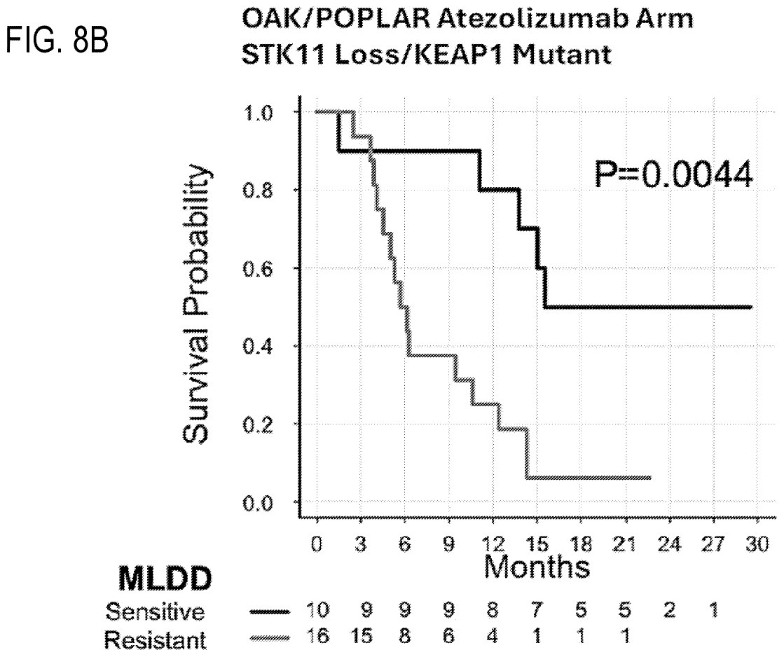

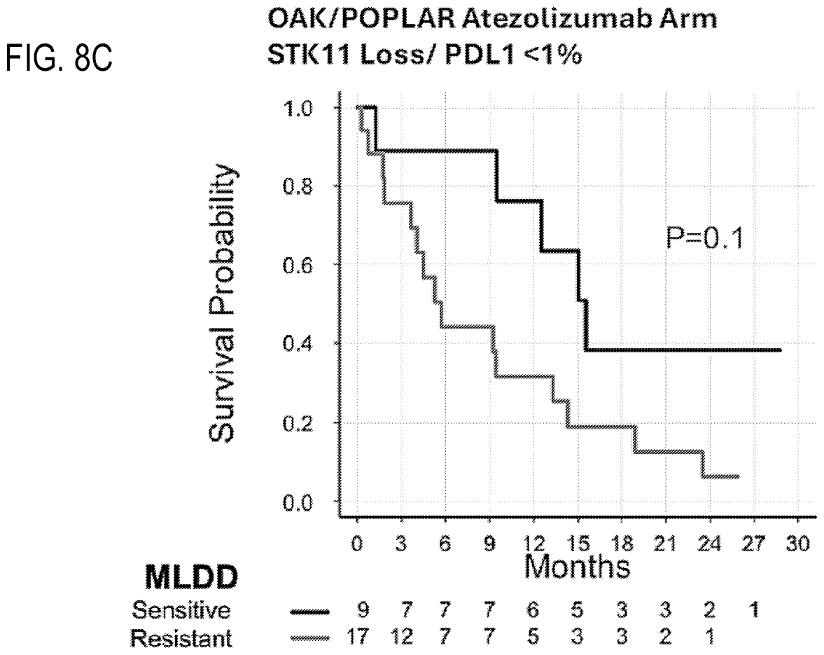

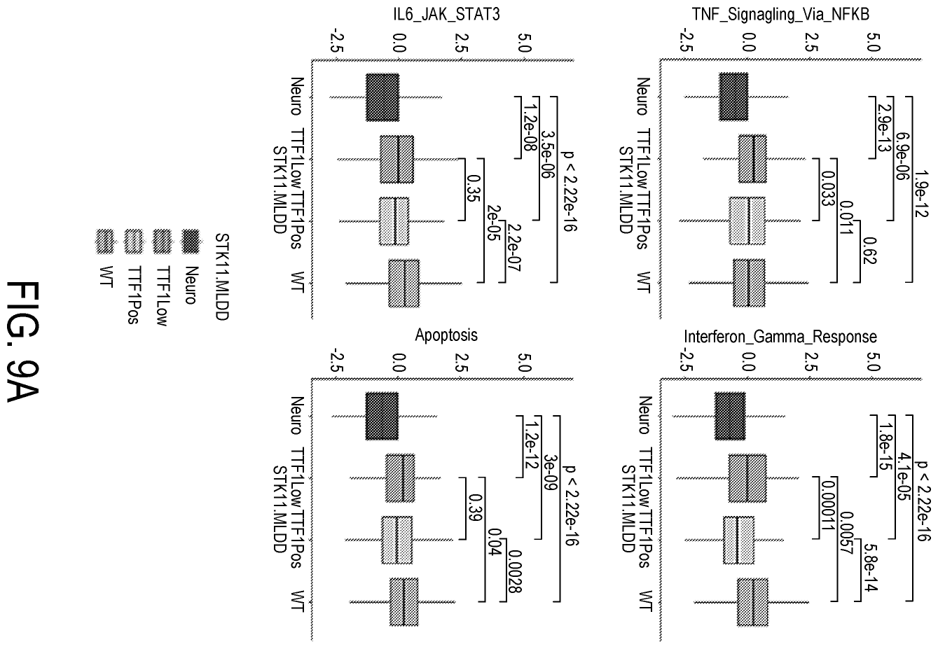

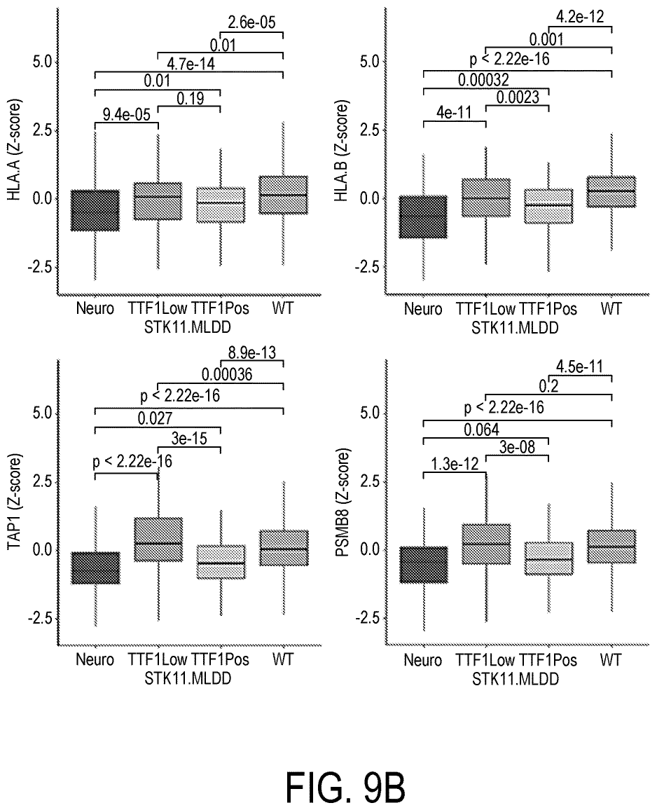

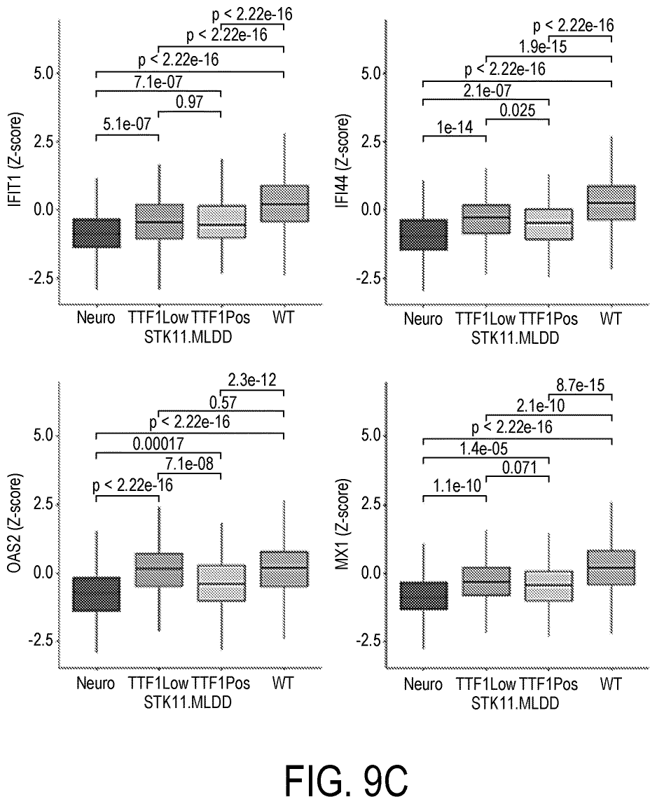

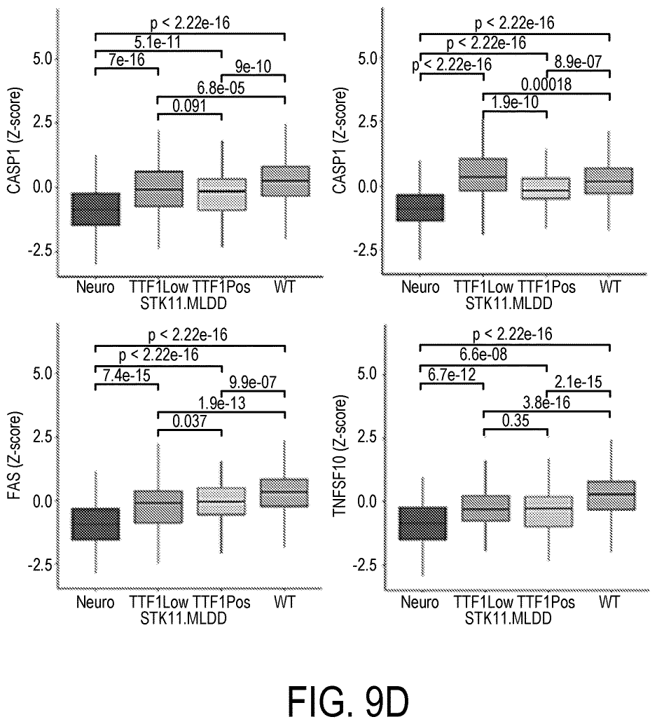

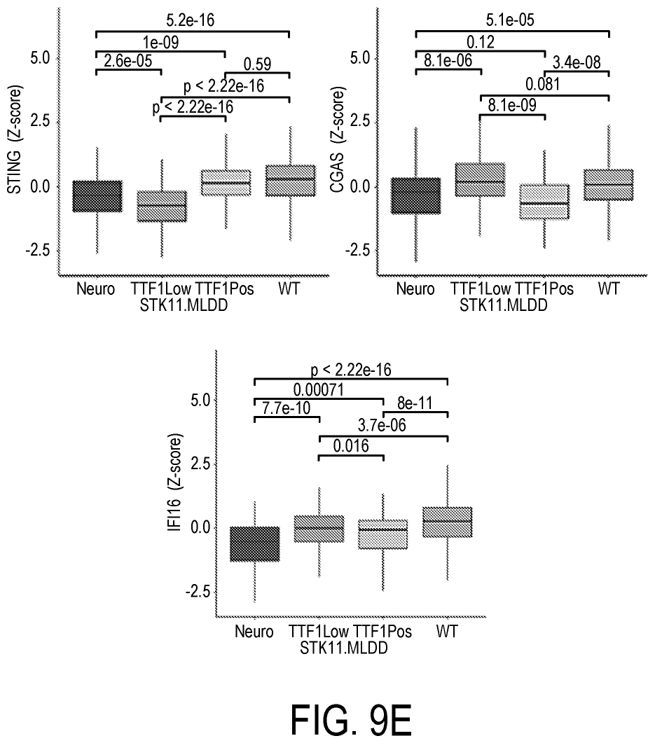

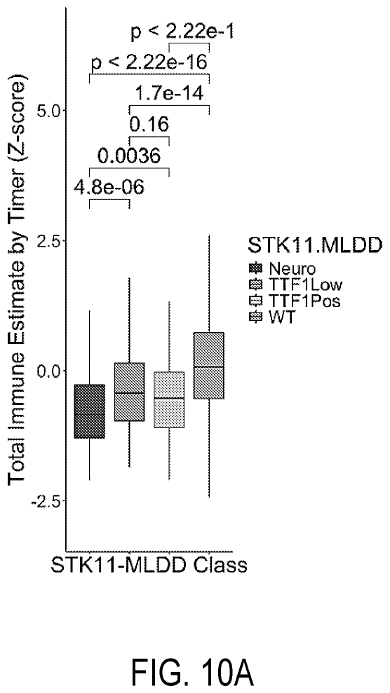

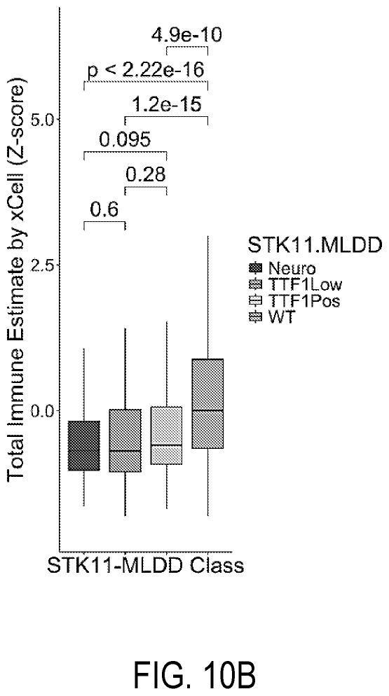

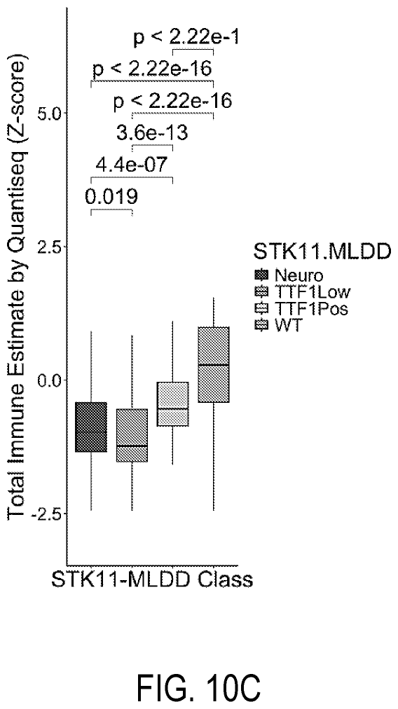

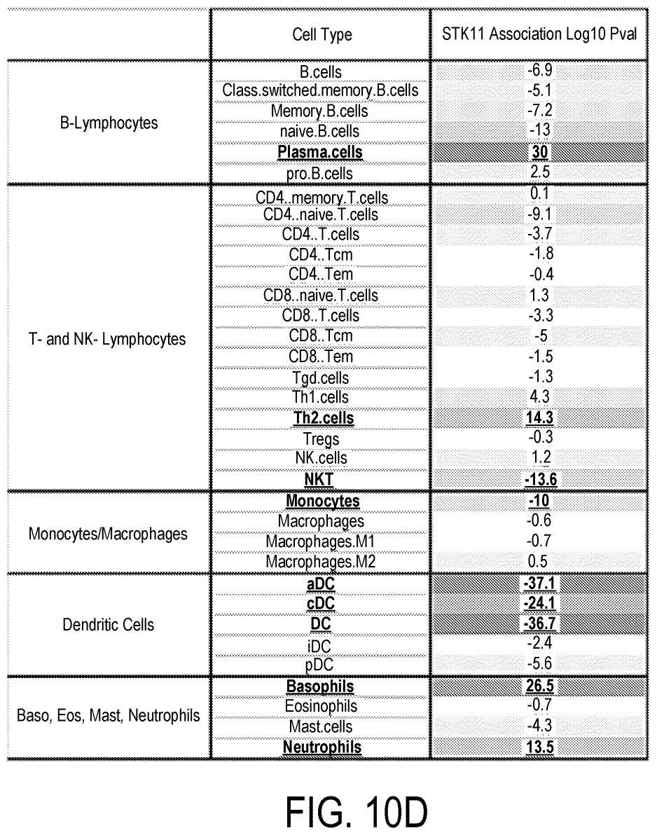

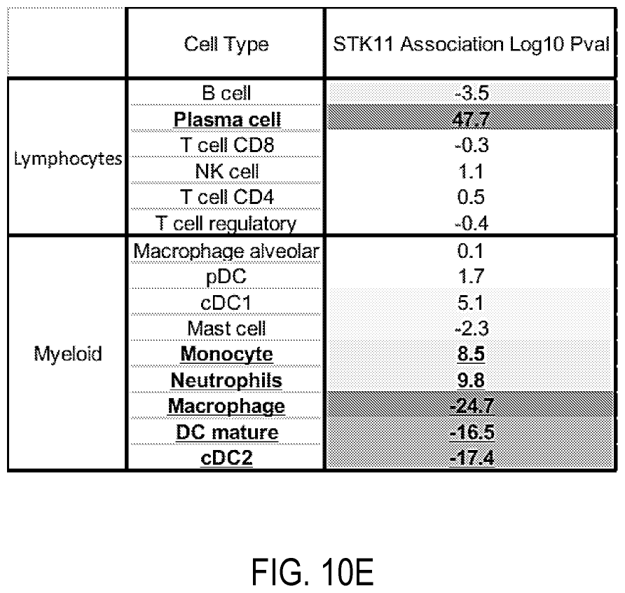

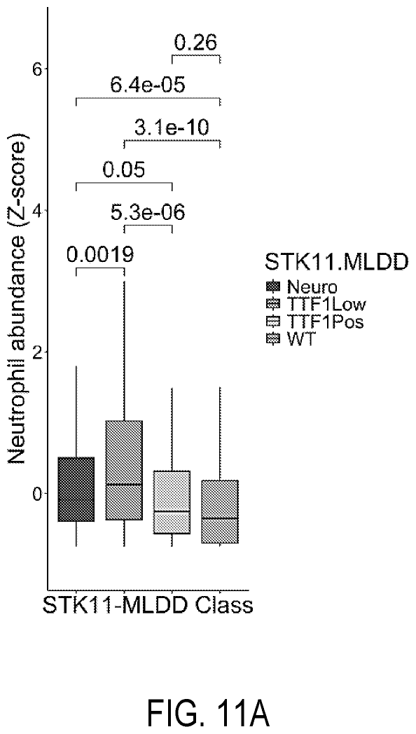

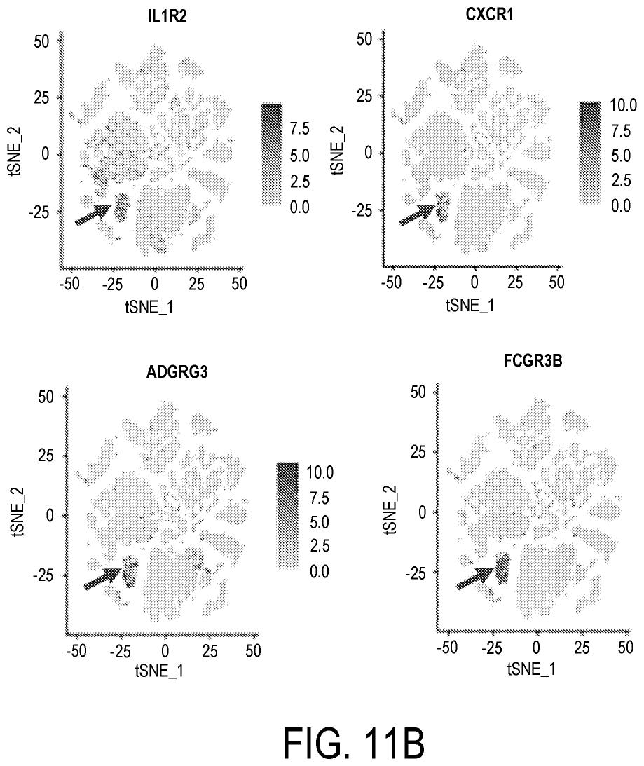

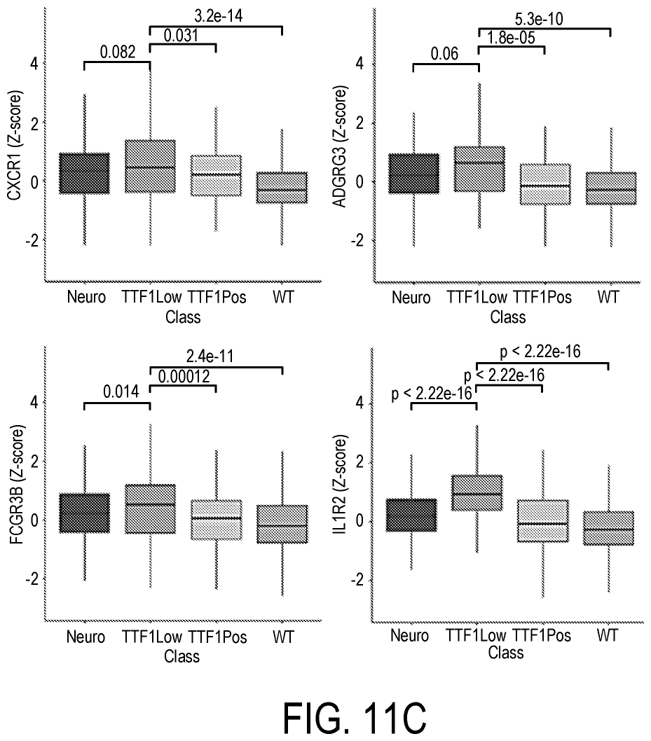

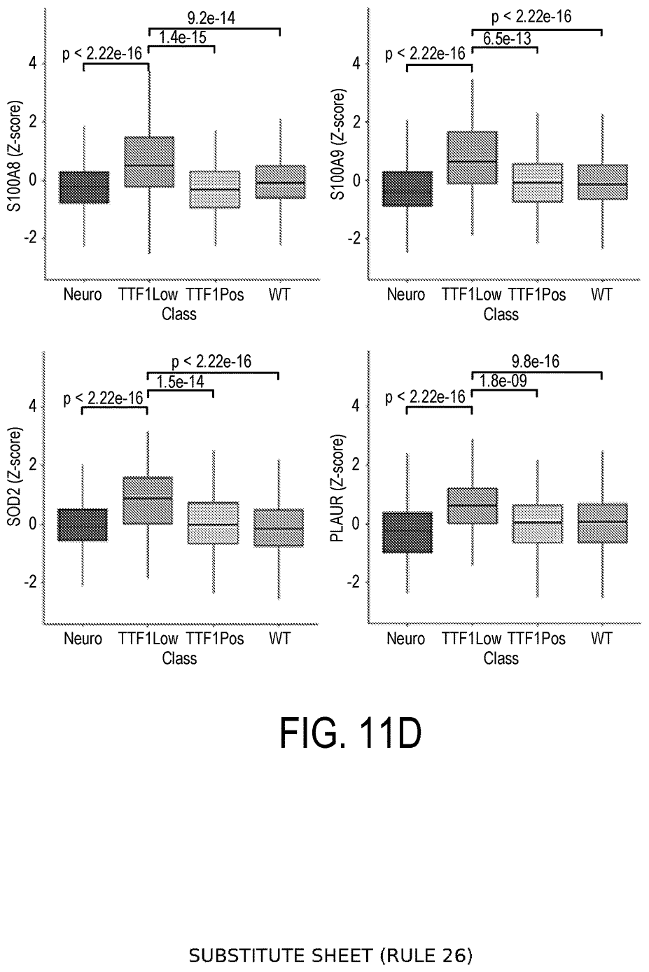

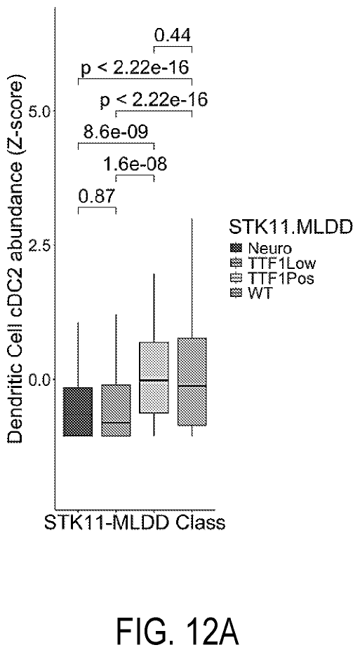

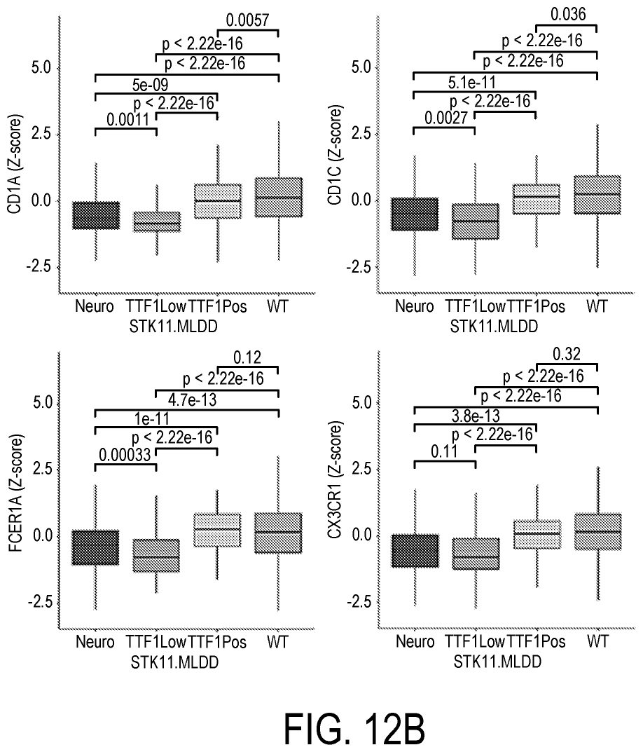

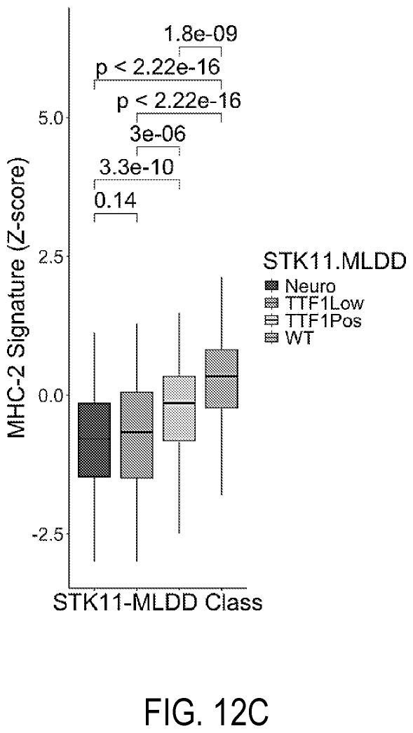

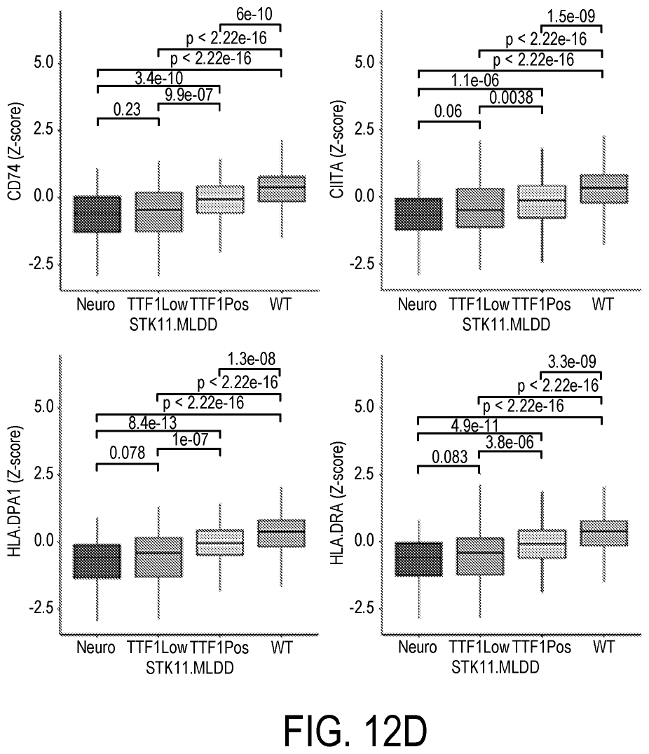

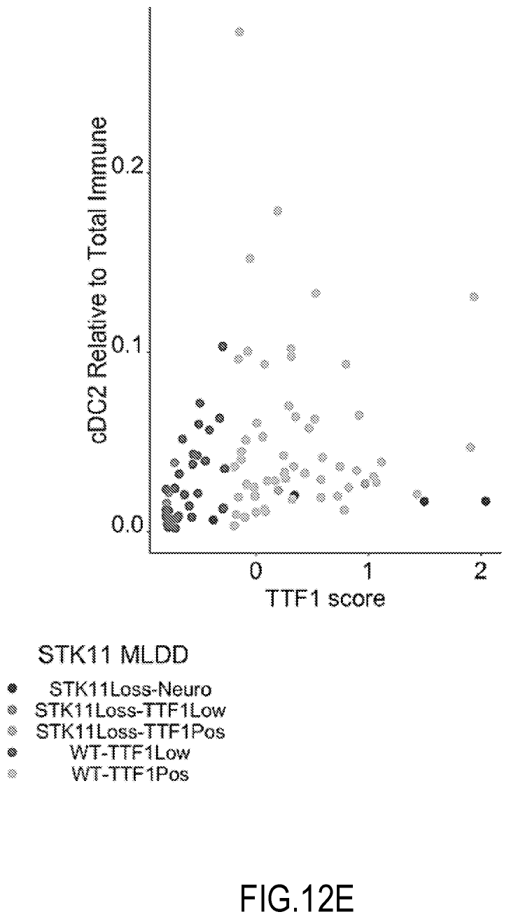

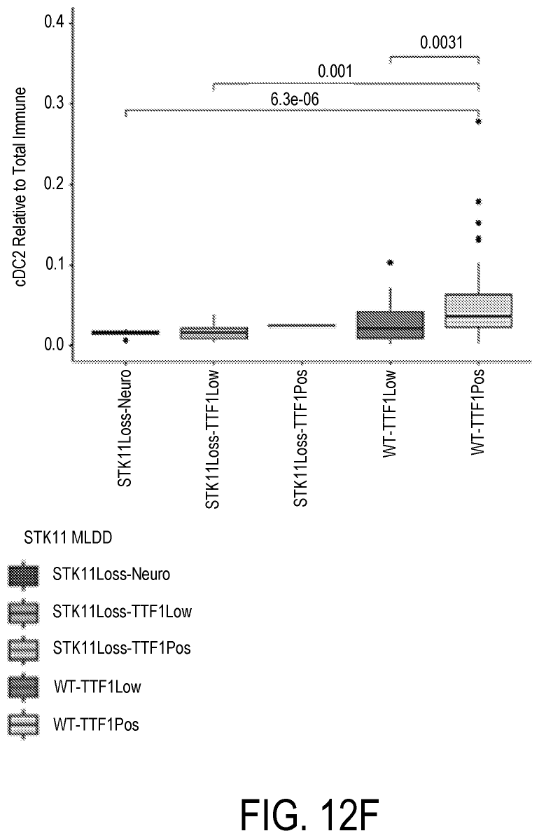

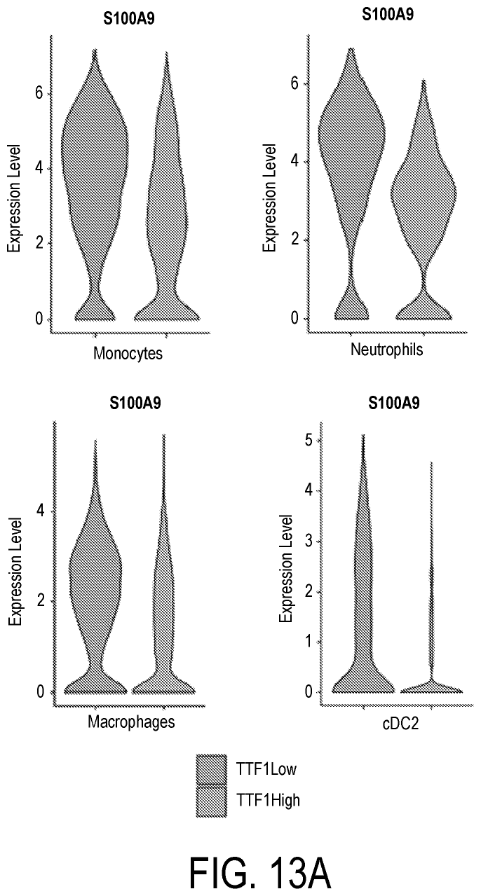

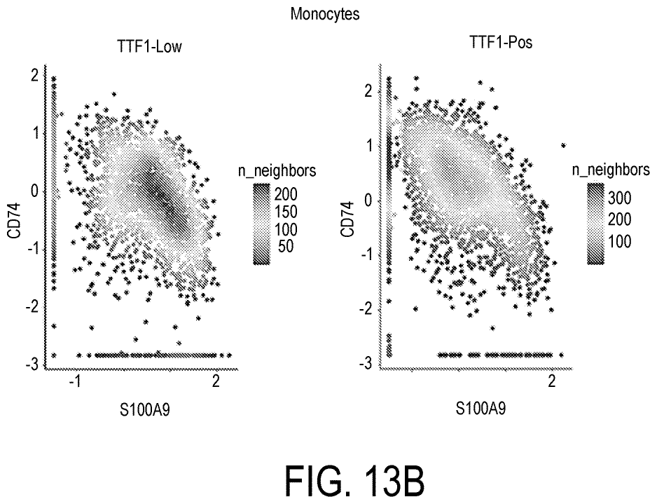

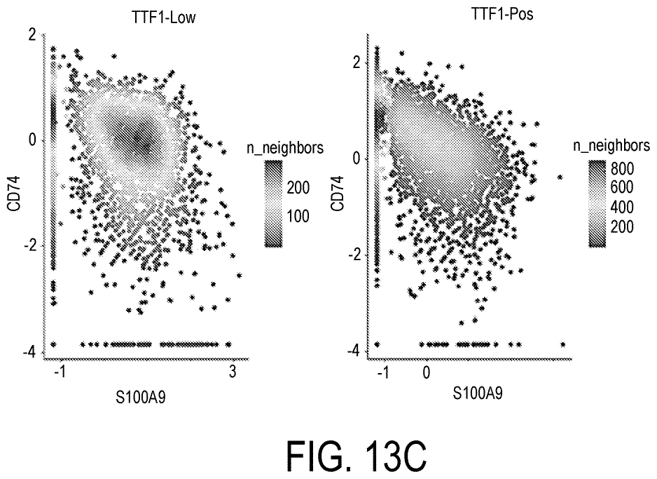

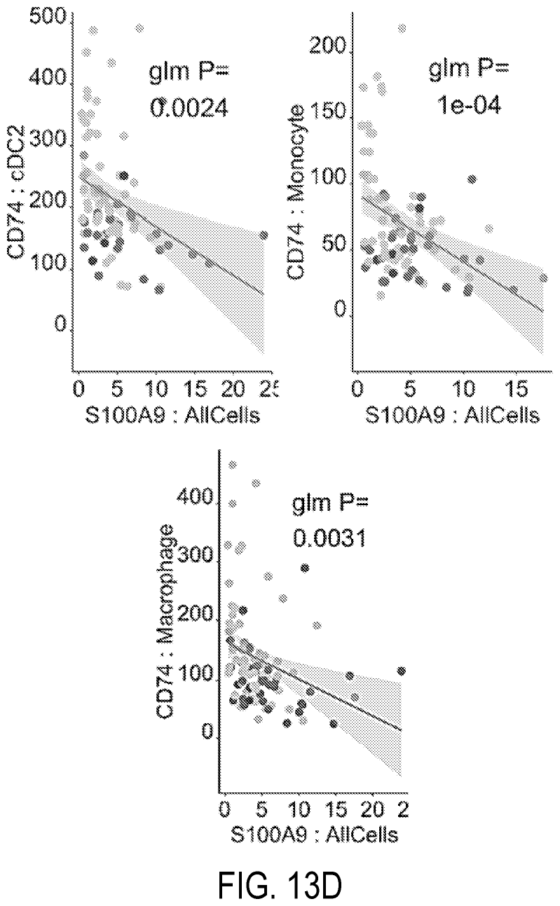

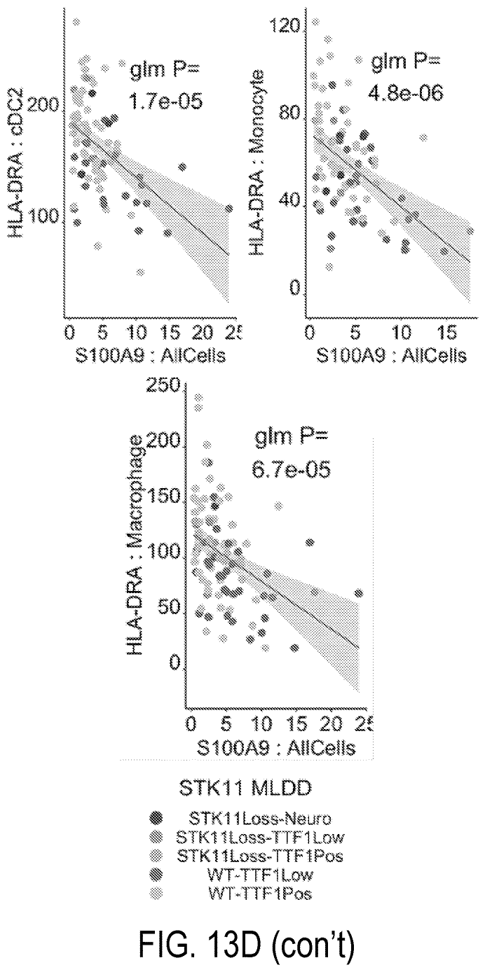

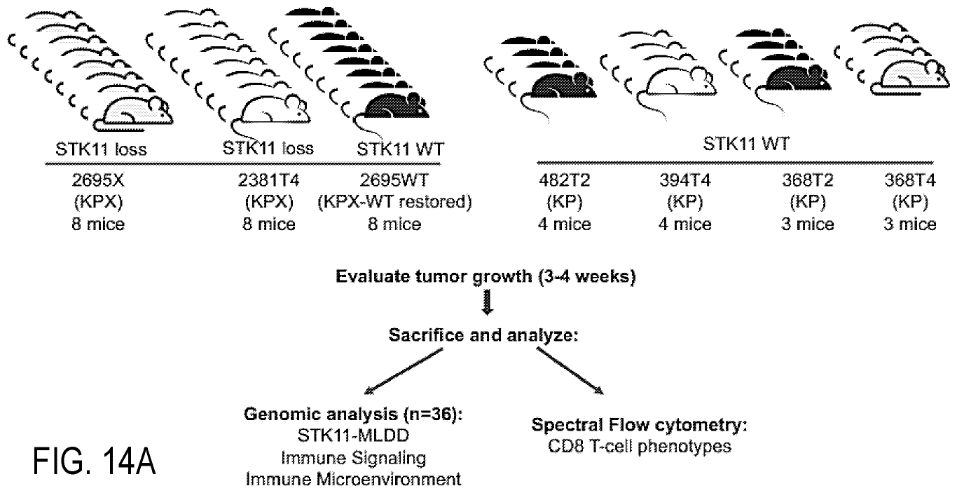

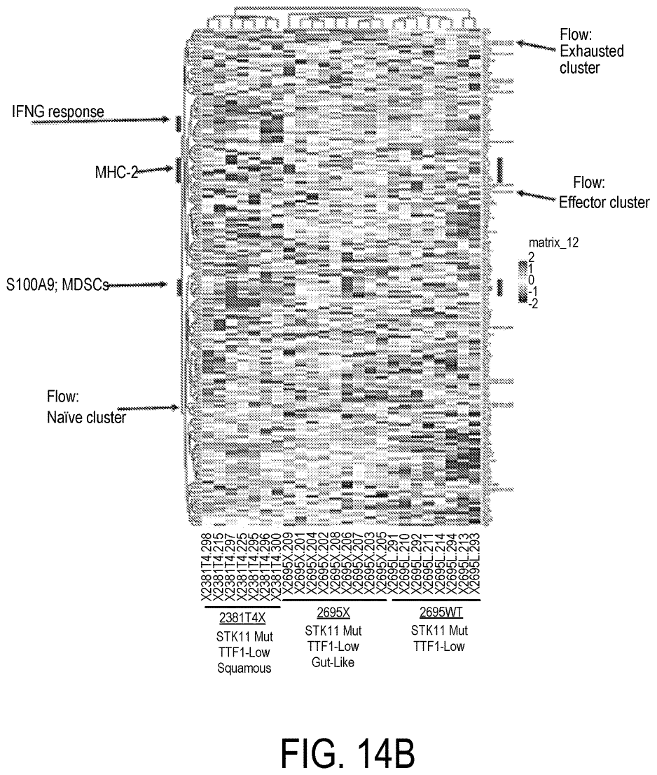

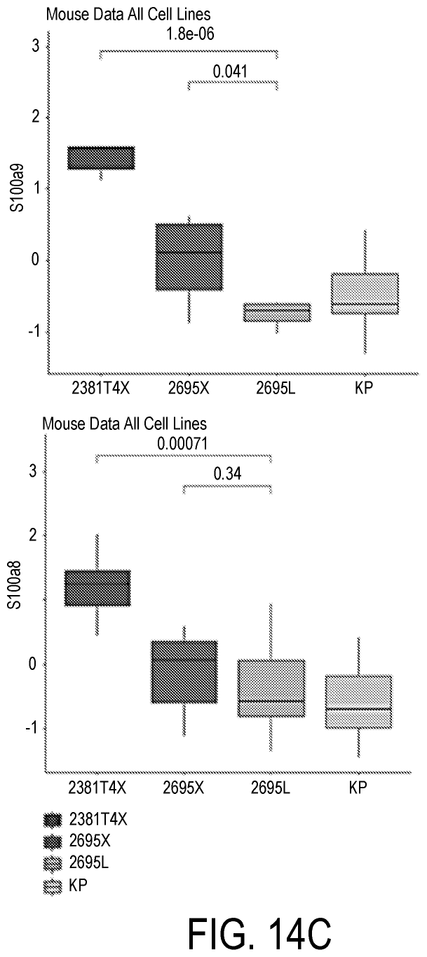

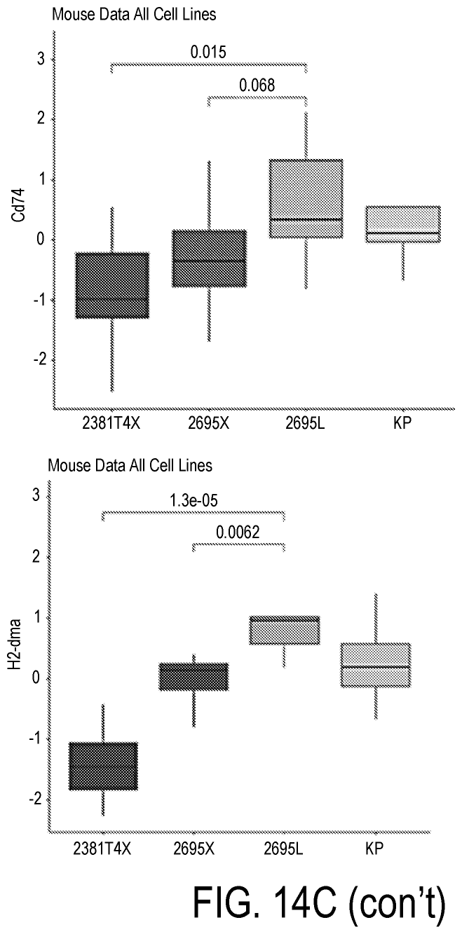

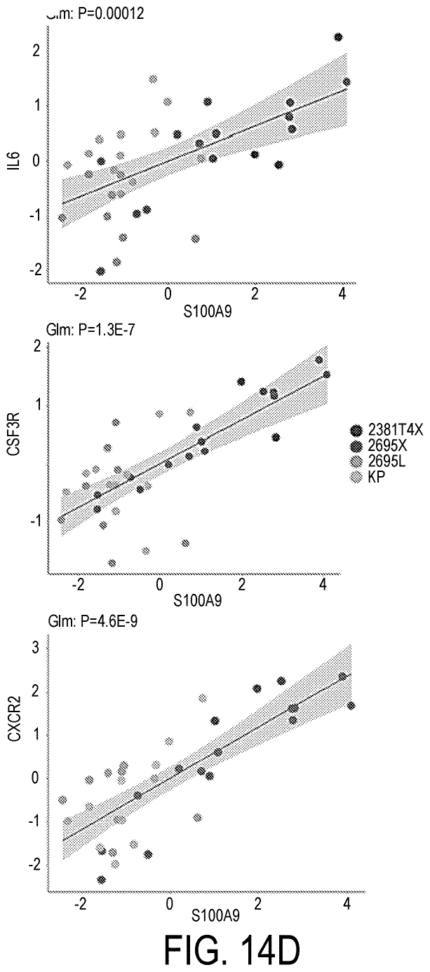

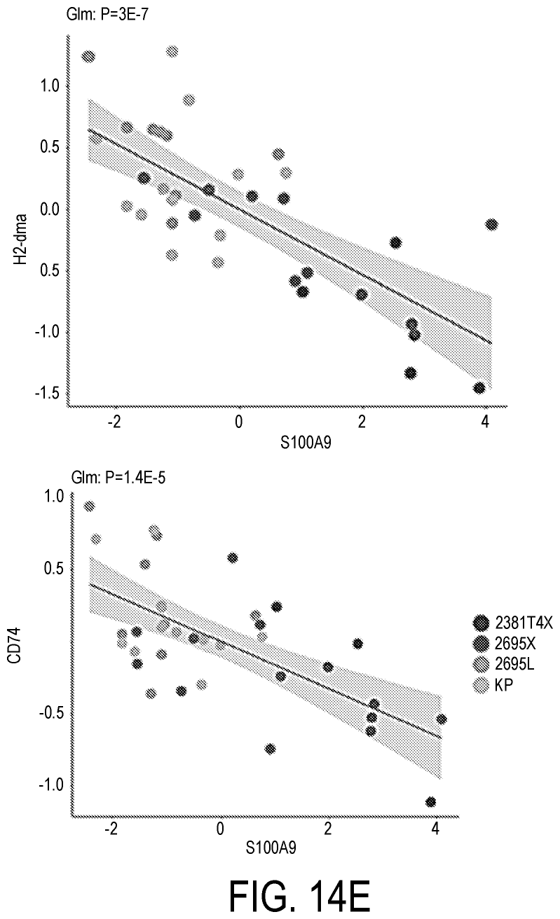

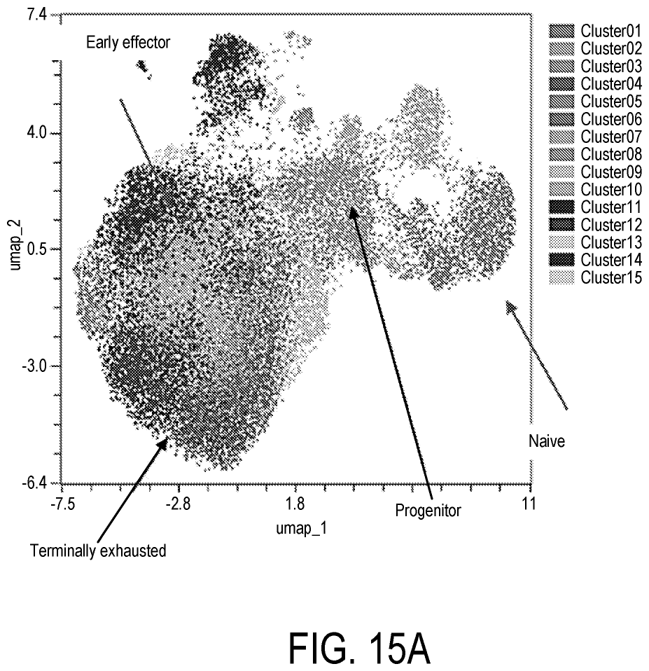

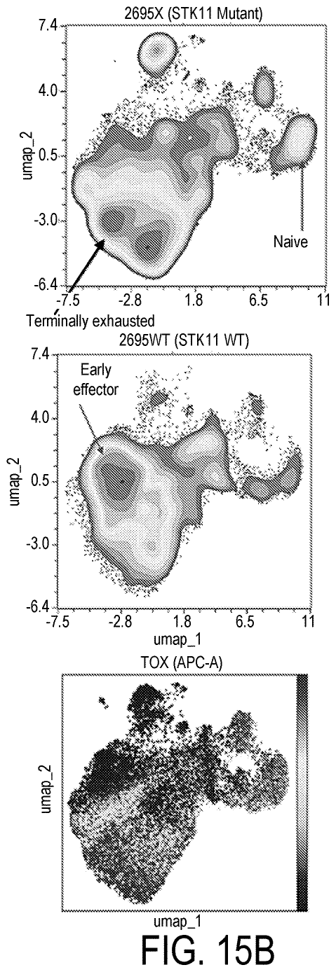

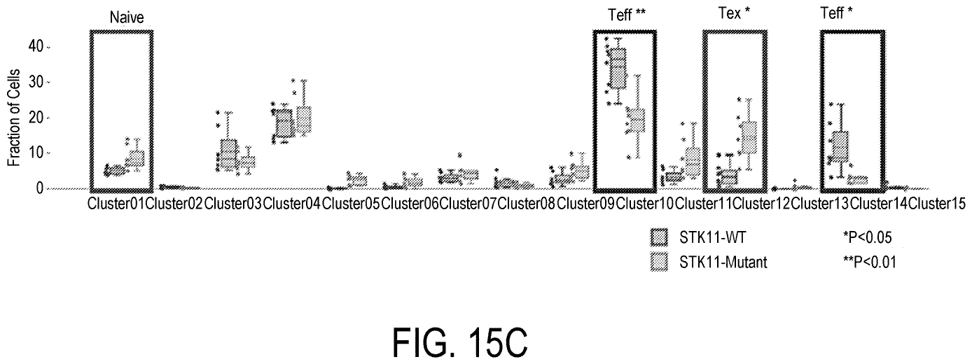

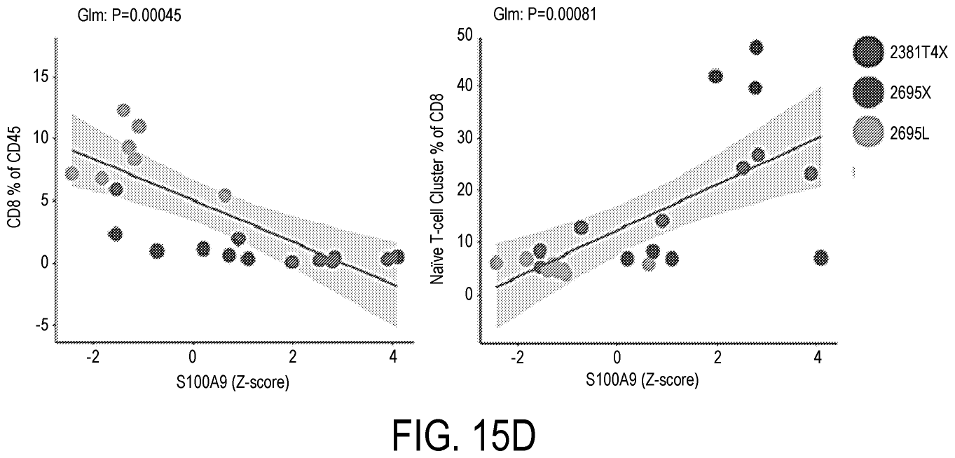

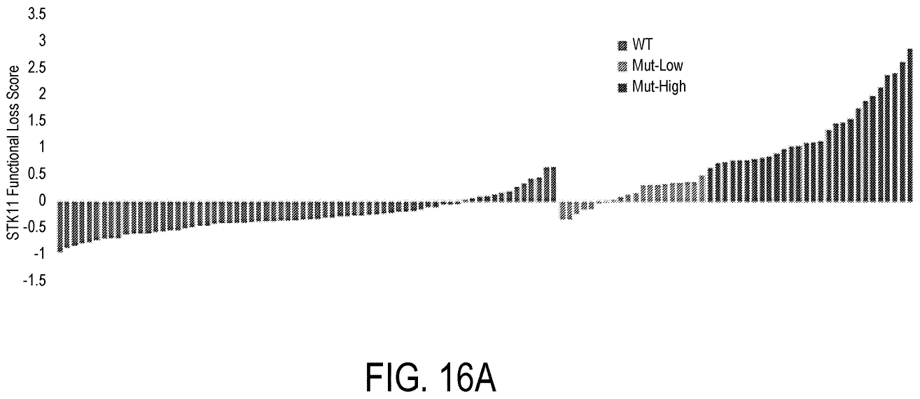

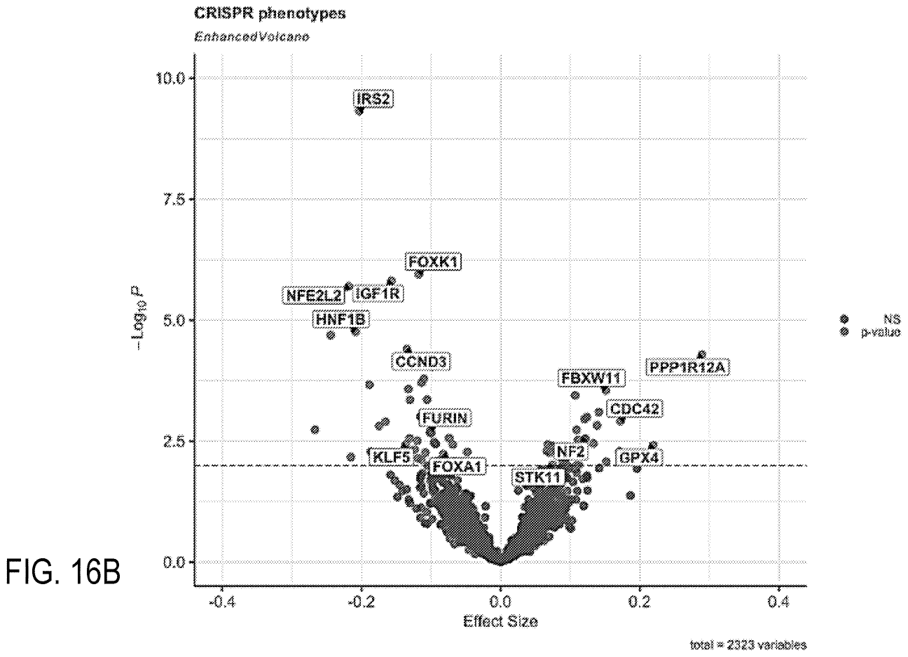

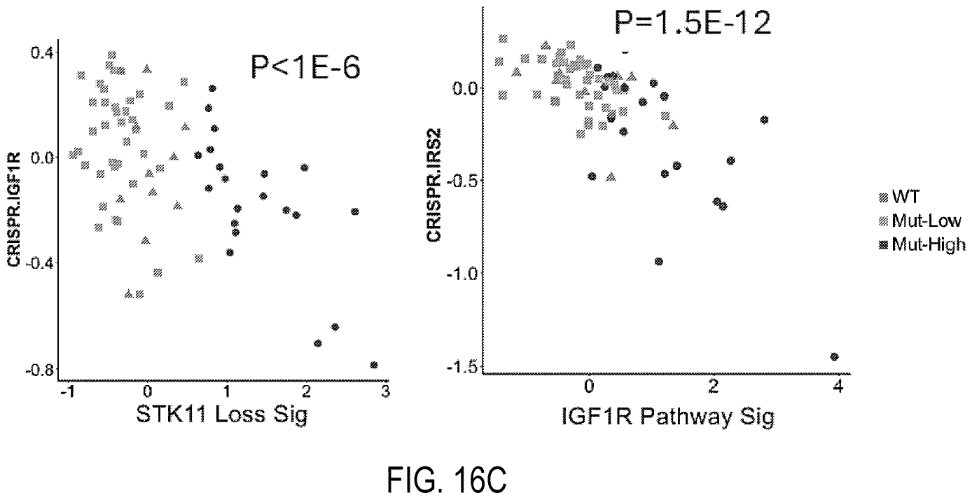

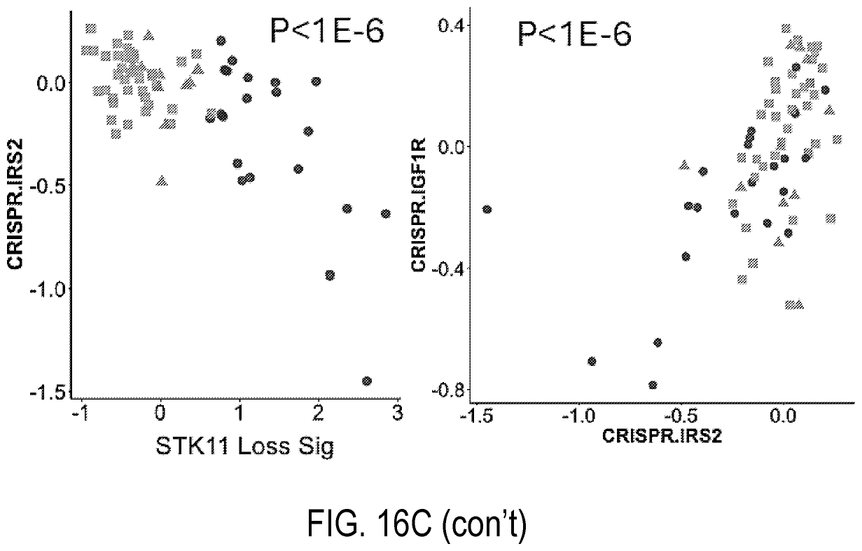

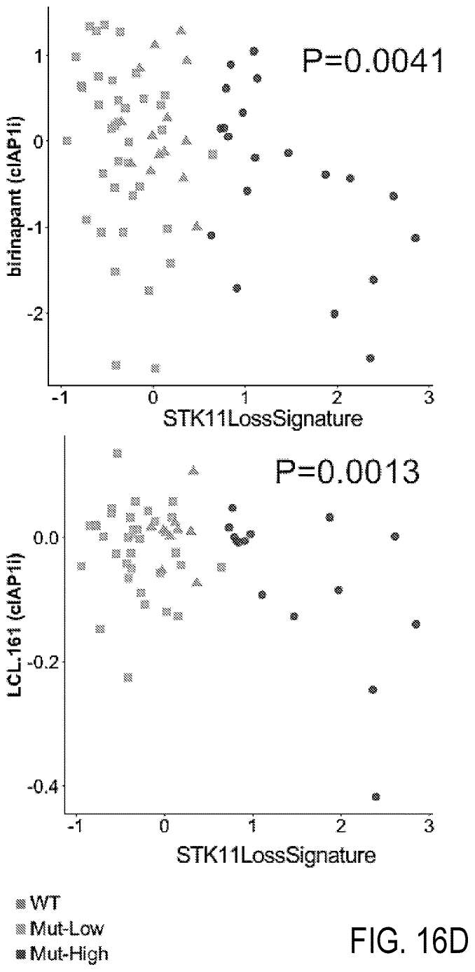

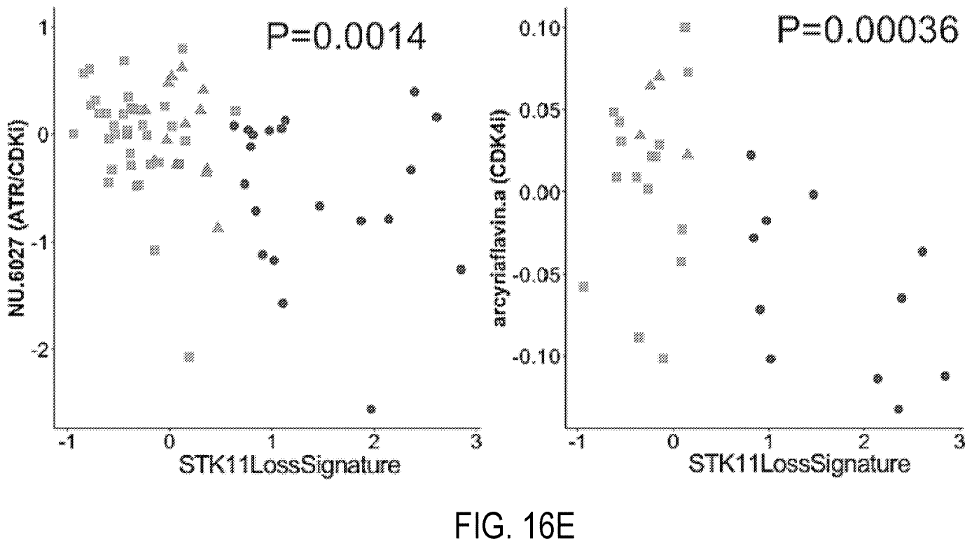

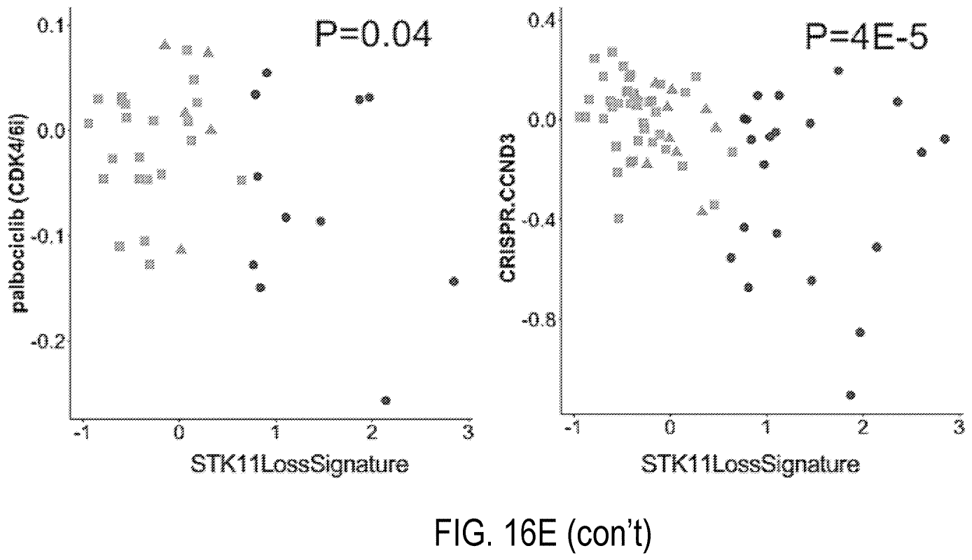

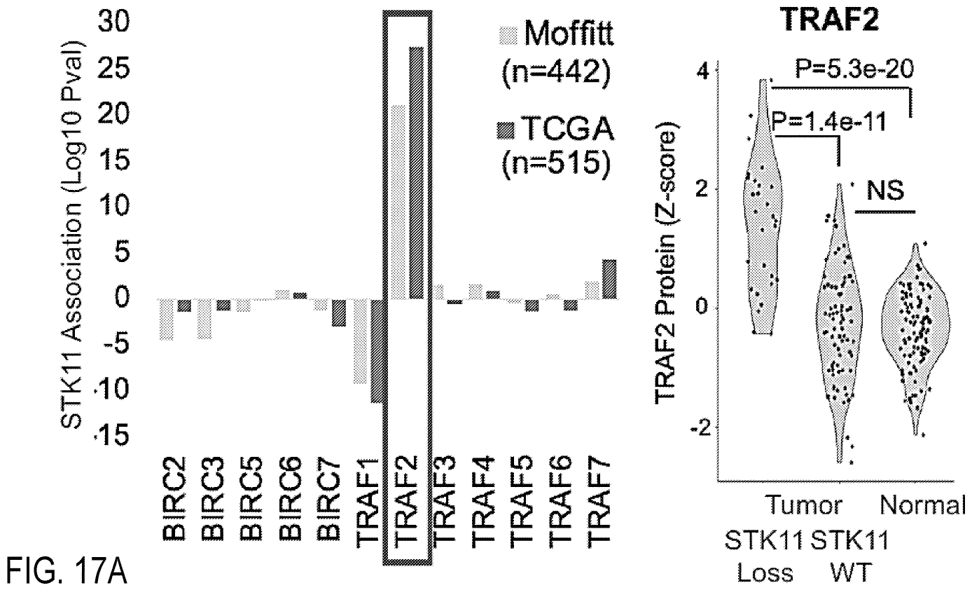

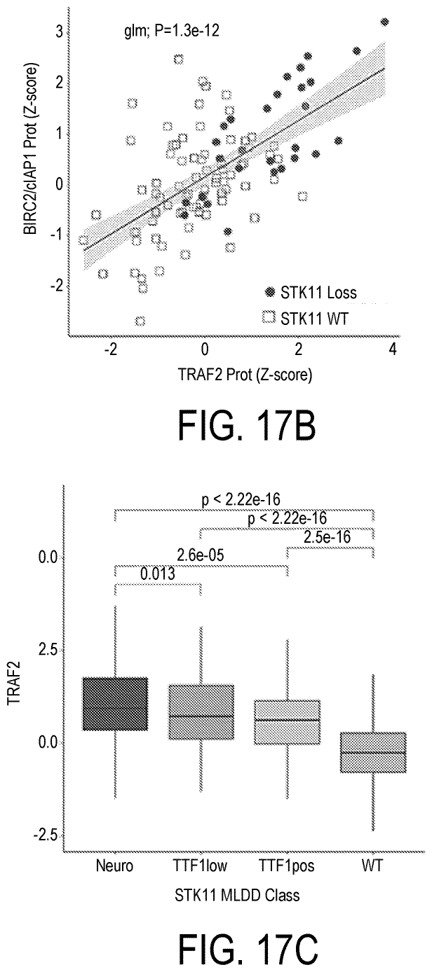

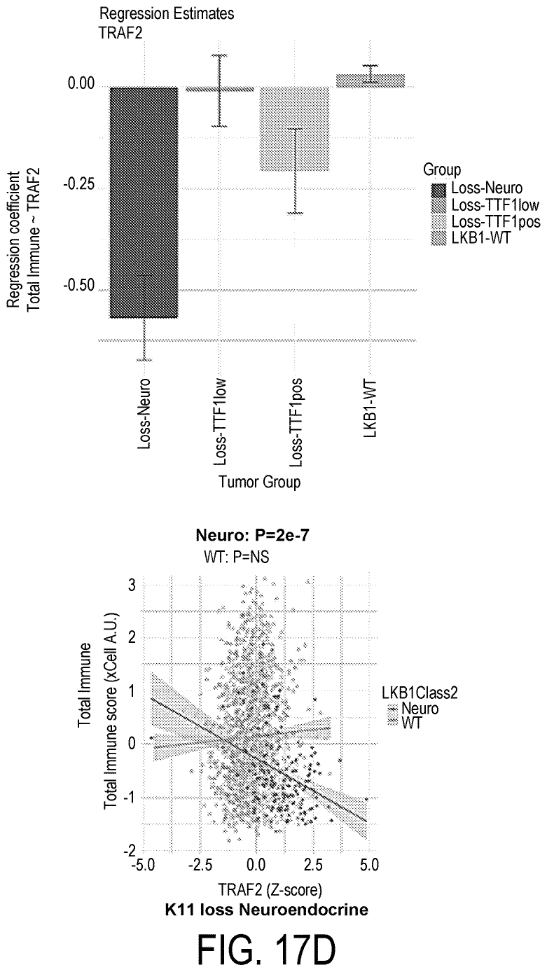

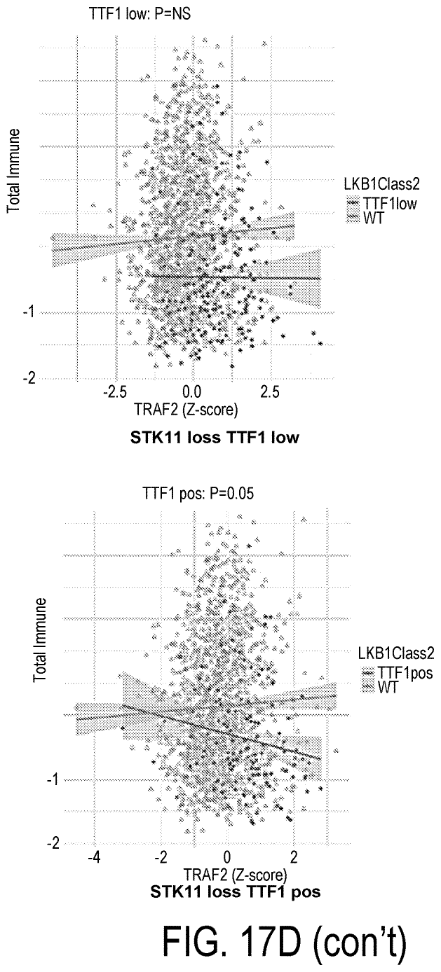

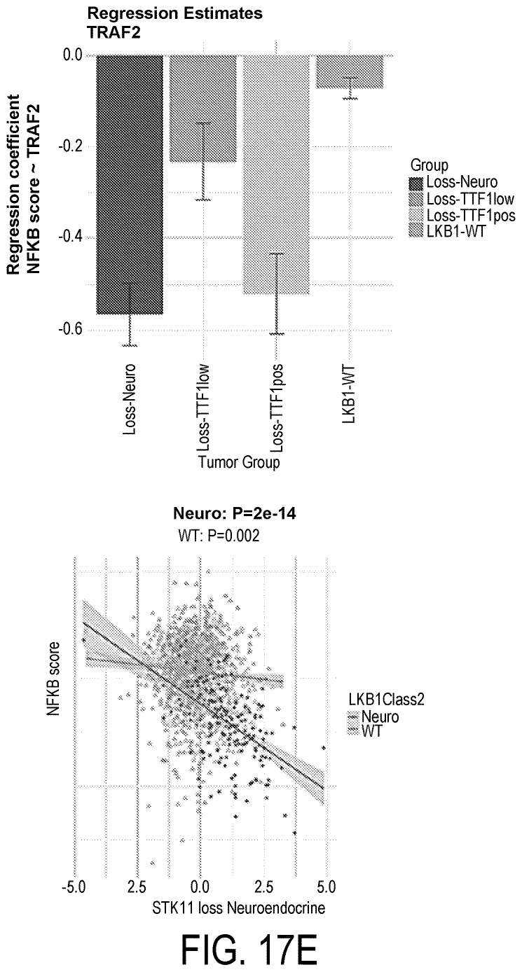

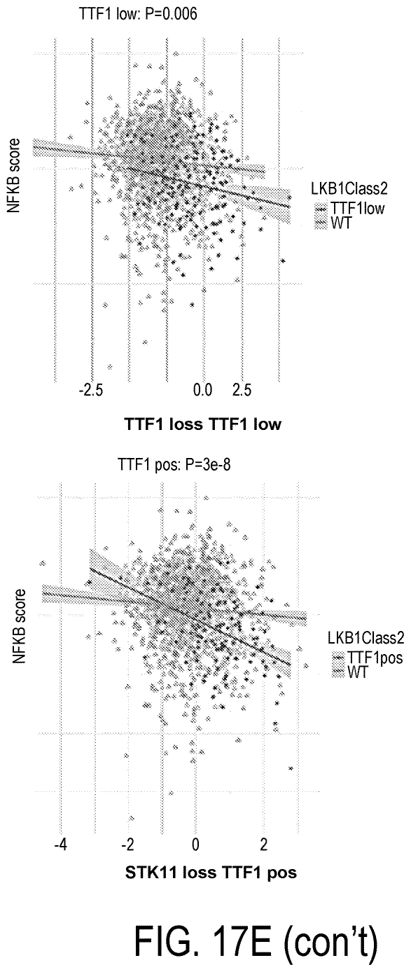

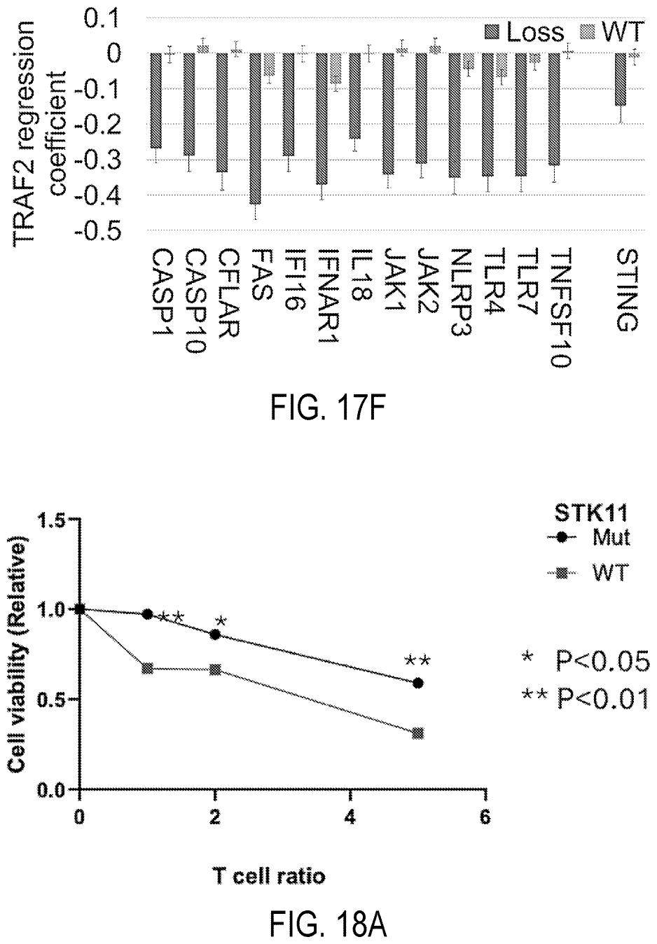

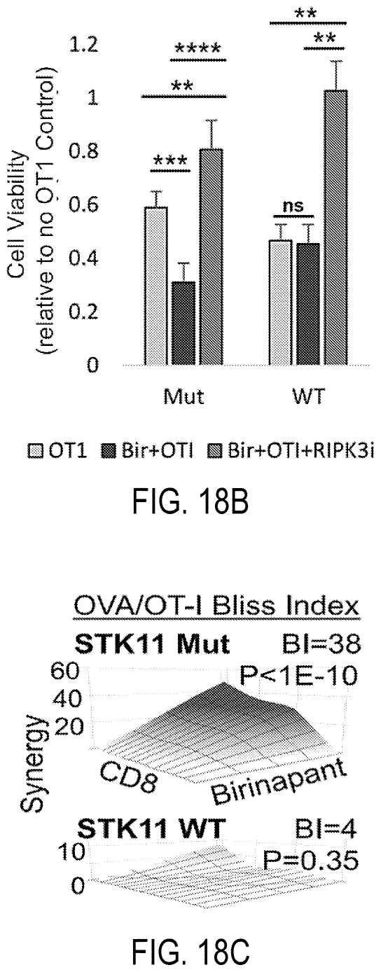

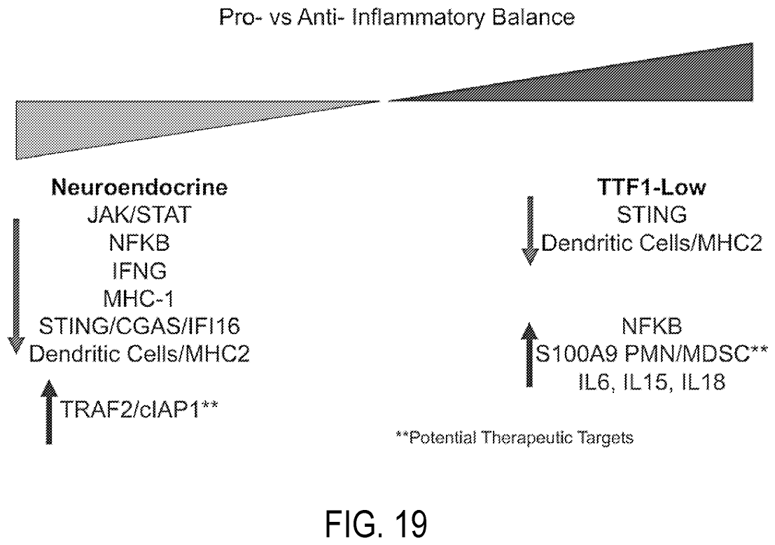

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (1) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). “Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. CDR3s are most variable, of which the tertiary structure determines antigen recognition of an antibody. Framework Regions (FRs) refer to amino acid sequences interposed between CDRs.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part. In some embodiments, the cancer used herein refers to lung cancer (e.g., non-small cell lung cancer (NSCLC), Small cell lung cancer (SCLC), lung carcinoid tumor, or pancoast tumor).

A “control” is an alternative subject or sample used in an experiment for comparison purpose. A control can be “positive” or “negative.” In some embodiments, the term “control” or “reference control” refers to a level in a sample derived from a cancer free or healthy individual, a sample taken at a different stage in disease development, a reference database, or a sample from a general or study population.

As used herein, the terms “determining,” “measuring,” and “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

As used herein the term “encoding” refers to the inherent property of specific sequences of nucleotides in a nucleic acid, to serve as templates for synthesis of other molecules having a defined sequence of nucleotides (i.e., rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPSTM technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

An “expression product” or “gene product” as used herein refer to one or more amino acids, including a peptide, an oligopeptide, a polypeptide, or variations thereof, generated when a gene is transcribed and translated. The “expression product” or “gene product” also refer to a protein molecule that is the product of the expression of a gene, through which the gene influences or regulates cell functions, including but not limited to motility, development, and/or metabolism. The intermediate molecule between the gene and the protein molecule is an RNA molecule (i.e., messenger RNA (mRNA), transfer RNA (RNA), and ribosomal RNA (rRNA)) that is the product of gene expression and further translated into the protein molecule.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

The term “increased” or “increase” as used herein generally means an increase by a statically significant amount; for the avoidance of any doubt, “increased” means an increase of at least 10% as compared to a reference level or a control, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level or a control, or at least about a 2-fold, or at least about a 3-fold, or at least about a 4-fold, or at least about a 5-fold or at least about a 10-fold increase, or any increase between 2-fold and 10-fold or greater as compared to a reference level or a control.

The term “reduced”, “reduce”, “reduction”, or “decrease” as used herein generally means a decrease by a statistically significant amount. However, for avoidance of doubt, “reduced” means a decrease by at least 10% as compared to a reference level or a control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference or control sample), or any decrease between 10-100% as compared to a reference level or a control.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “subject” or “host” refers to any individual who is the target of administration or treatment. The subject can be a vertebrate, for example, a mammal. Thus, the subject can be a human. The term “patient” refers to a subject under the treatment of a clinician, e.g., physician. The subject can be either male or female.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g., cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular biomarker is disclosed and discussed and a number of modifications that can be made to a number of molecules including the biomarker are discussed, specifically contemplated is each and every combination and permutation of biomarker and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### Methods of Diagnosing and Treating Cancers

STK11 loss affects lineage plasticity in murine models and multi-lineage de-differentiation (MLDD) phenotypes in patients. MLDD is an established mechanism of resistance to oncogene targeted therapies and affects prognosis. However, interactions between MLDD, the immune microenvironment, and immunotherapy response are not well characterized. Disclosed herein are biomarkers associated uses of STK11-MLDD as a clinically actionable biomarker based on measured gene expression corresponding to three key tumor phenotypes.

At present, there is a knowledge gap in using STK11-MLDD as classifier. While STK11 mutations have been linked to immunotherapy resistance, some STK11-mutant tumors can still respond well to immunotherapy, and loss of STK11 also seems to affect prognosis and response to chemotherapy. The instant disclosure shows that STK11-MLDD classifier can identify a subset of STK11-mutant tumors that are immunotherapy responsive, and also accurately identifies functional loss of STK11 in tumors in which no mutations were detected on sequencing. Further, identification of differentiation phenotypes is currently dependent on Histopathological evaluation, which is an imperfect tool and not well suited for clinical biomarker development. The present disclosure provides STK11-MLDD as a clinically relevant and actionable biomarker for the standardized prediction and classification of patient responsiveness to cancer mediations, including but limited to immunotherapeutic agents.

In some aspects, disclosed herein is a method of predicting responsiveness of a subject having a cancer to an immunotherapeutic agent, comprising obtaining a biological sample from the subject, determining a level of one or more biomarkers in the biological sample, wherein the one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1, determining an expression score for each of the one or more biomarkers in the biological sample relative to a reference control, determining a mean expression score by averaging the one or more expression scores of the one or more biomarkers, and predicting the subject as responsive to the immunotherapeutic agent if the mean expression score is lower than a pre-determined threshold.

The present disclosure provides a scoring method using a “z-score”. As used herein, a “z-score” has been contemplated to refer to the calculation of a score based on an average of samples. Further, the z-score of any disclosed aspect requires knowledge of a reference sample range, mean, and standard deviation. The present disclosure also provides a scoring method using a “singscore”. As used herein, a “singscore” has been contemplated to refer to the calculation of a score based on a single sample, which is advantageous for commercial development when applied to sample analyzed by RNAseq. It should be understood that the “z-score” and “singscore” approaches are equivalent in reference to detecting STK11 mutations.

The present disclosure also provides methods of calculating a functional biomarker ‘score’ using a Biomarker Gene Set. Herein, a “Biomarker Gene Set” refers a pre-specified list of genes that have been determined to be associated with a biologic phenotype within a particular, such as for example lung cancer. These genes are typically strongly correlated in expression, such that their expression data can be integrated to yield a single functional score. Two computational methods for calculating such scores have been tested, and these two approaches yield concordant results. Both approaches can successfully predict tumor characteristics including STK11 mutational status. Herein, method 1 is referred to as the ‘mean Z-score’ approach, which involves calculation of the Z-score for each gene comprising the Biomarker Gene Set, and then averaging these together to give a single score. Herein, method 2 is referred to as the ‘SingScore’ approach, which involves a single sample gene set enrichment calculation comparing the expression level of each Biomarker Gene Set with the global range of mRNA expression across an individual sample.

Each approach has advantages for certain applications. The ‘mean Z-score’ approach is most useful in analyzing large datasets when all data have already been collected, as well as integrating several different datasets. The ‘SingScore’ approach yields a biomarker score based solely on the data contained in a single sample, and can be readily applied to RNA sequencing data, such as that obtained from standard-of-care molecular testing in lung cancer. For prediction of STK11 mutational status, the ‘mean Z-score’ approach is highly predictive, with AUC of 0.88, while the ‘singscore’ approach is equally predictive, with AUC of 0.85.

Method 1: the ‘mean Z-score’ approach. The expression values for each gene in the Biomarker Gene Set is determined relative to the mean and standard deviation of an appropriate set of reference control samples. The ‘mean Z-score’ of a specified Biomarker Gene Set is then the mean of the Z-scores for each component gene: shown below for GeneSetX, comprising n genes. The Biomarker Mean Z-Score is a continuous variable, which can be converted to a binary classifier using a numeric cut-point threshold: a sample with a score greater than this cut-point will be classified as ‘positive’, while a sample with score below the cut-point will be classified as ‘negative’.

$$Z . {score}_{geneX . n} = ( {Expression}_{geneX . n} - {Mean}_{geneX} ) / ( Standard . {Deviation}_{geneX} )$$

$$Mean . Z . {score}_{GeneSetX} = Mean ( Z . {score}_{geneX \text{.1}} , Z . {score}_{geneX \text{.2}} , Z . {score}_{geneX \text{.3}} , … Z . {score}_{geneX . n} )$$

$$Binary . Mean . Z . {score}_{GeneSetX} = IF ( Mean . Z . {score}_{GeneSetX} > Z . Cut . {Point}_{GeneSetX} ) THEN ‘ Positive ’ ; ELSE IF ( mean . Z . {score}_{GeneSetX} < Z . Cut . {Point}_{GeneSetX} ) THEN ‘ Negative ’$$

Method 2: the ‘SingScore’ approach. SingScore is a simple single-sample gene signature scoring method that uses rank-based statistics to analyze the sample's gene expression profile. It scores the expression activities of gene sets at a single-sample level. This approach uses the ‘singscore’ package (Foroutan M, Bhuva D, Lyu R, Horan K, Cursons J, Davis M (2018). “Single sample scoring of molecular phenotypes.” BMC bioinformatics, 19 (1), 404. doi:10.1186/s12859-018-2435-4; and Bhuva D, Cursons J, Davis M (2020). “Stable gene expression for normalisation and single-sample scoring.” Nucleic Acids Research, 48 (19), e113. doi:10.1093/nar/gkaa802) in RStudio and calculates a single sample gene set enrichment score using the genes comprising the Biomarker Gene Set.

A non-limiting example of a code using RStudio includes:

```
{
BiocManager::install(″singscore″)
library(singscore)
rankData <− rankGenes(Sample.PtID.X)
  #Ranks genes in by sample
Singscore_{GeneSetX} <− simpleScore(rankData, upSet = GeneSetX)
$TotalScore
  #Performs the gene set scoring from the rank data
}
Binary.Singscore_{GeneSetX} =
  if( Singscore_{GeneSetX} > Singscore.Cut.Point_{GeneSetX} ) THEN ‘Positive’;
ELSE
  if( Singscore_{GeneSetX} < Singscore.Cut.Point_{GeneSetX}) THEN ‘Negative’
```

In some embodiments, the expression score is a z-score. In some embodiments, the expression score is a singscore.

Accordingly, in some aspects, disclosed herein is a method of predicting responsiveness of a subject having a cancer to an immunotherapeutic agent, comprising obtaining a biological sample from the subject, determining a level of one or more biomarkers in the biological sample, wherein the one or more biomarkers are one or more RNAs, or an expression product thereof, of the genes selected from the group consisting of AVP I1, BAG1, CPS1, DUSP4, FGA, GLCE, HAL, IRS2, MUC5AC, PDE4D, PTP4A1, RFK, SIK1, TACC2, TESC, and TFF1, determining a z-score for each of the one or more biomarkers in the biological sample relative to a reference control, determining a mean z-score by averaging the one or more z-scores of the one or more biomarkers, and predicting the subject as responsive to the immunotherapeutic agent if the mean z-score is lower than 0.5.

Accordingly, in some aspects, disclosed herein is a method of predicting responsiveness of a subject having a cancer to an immunotherapeutic agent, comprising obtaining a biological sample from the subject, determining a level of one or more biomarkers in the biological sample, wherein the one or more biomarkers are one or more RNAs, or an expression product thereof, of the genes selected from the group consisting of AVP I1, BAG1, CPS1, DUSP4, FGA, GLCE, HAL, IRS2, MUC5AC, PDE4D, PTP4A1, RFK, SIK1, TACC2, TESC, and TFF1, determining a singscore for each of the one or more biomarkers in the biological sample relative to a reference control, determining a singscore by integrating the one or more singscores of the one or more biomarkers, and predicting the subject as responsive to the immunotherapeutic agent if the singscore is lower than 0.5.

In some aspects, disclosed herein is a method of predicting responsiveness of a subject having a cancer to an immunotherapeutic agent, comprising obtaining a biological sample from the subject, determining a level of one or more biomarkers in the biological sample, wherein the one or more biomarkers comprise one or more RNAs (e.g., mRNAs), or an expression product thereof, of the genes selected from the group of genes representing the STK11 Functional Loss Classifier (including, for example, AVPI1, BAG1, CPS1, DUSP4, FGA, GLCE, HAL, IRS2, MUC5AC, PDE4D, PTP4A1, RFK, SIK1, TACC2, TESC, and TFF1), determining an expression score for each of the one or more biomarkers in the biological sample relative to a reference control, determining a STK11 Functional Loss Score by integrating the one or more expression scores of the one or more biomarkers, and predicting the subject as responsive to the immunotherapeutic agent if the STK11 Functional Loss Score is lower than a pre-determined threshold.

In some embodiments, the method further comprises administering to the subject a therapeutically effective amount of the immunotherapeutic agent. In some embodiments, the immunotherapeutic agent is an immune checkpoint inhibitor, including but not limited to a PD-1 inhibitor, a PD-L1 inhibitor, a PD-L2 inhibitor, or a CTLA-4 inhibitor. In some embodiments, the immunotherapeutic agent comprises Pembrolizumab, Nivolumab, Cemiplimab, Atezolizumab, Durvalumab, Ipilimumab, or Tremeliumumab. In some embodiments, the immunotherapeutic agent can be any therapeutic agent inhibiting the effects of the PD1 or CTLA4 immune checkpoint.

In some embodiments, the method further comprises predicting the subject as resistant to a first immunotherapeutic agent if the mean z-score is greater than 0.5. In some embodiments, the method further comprises administering to the subject a therapeutically effective amount of a second immunotherapeutic agent, with or without the co-administration of cytotoxic chemotherapy. In some embodiments, the second immunotherapeutic agent is an immune checkpoint inhibitor. In some embodiments, the second immunotherapeutic agent is administered in combination with another immunotherapeutic agent. Non-limiting examples of this combination of immunotherapeutic agents include, but are not limited to a PD-1 inhibitor combined with a CTLA-4 inhibitor, or a PD-L1 inhibitor combined with a CTLA-4 inhibitor, or a PD-1 inhibitor combined with a cIAP1 inhibitor, or a PD-L1 inhibitor combined with a cIAP1 inhibitor. In some embodiments, the PD-1 checkpoint inhibitor comprises Pembrolizumab, Nivolumab, Cemiplimab. In some embodiments, the PD-L1 checkpoint inhibitor comprises Atezolizumab or Durvalumab. In some embodiments, the cytotoxic chemotherapy comprises Carboplatin, Cisplatin, Paclitaxel, Gemcitabine, Pemetrexed, Nab-Paclitaxel, used alone or in combination. In some embodiments, the CTLA-4 inhibitor comprises Ipilimumab, or Tremeliumumab. In some embodiments, the cIAP1 inhibitor comprises birinapant, xevinapant, tolianapant, LC-161.

In some embodiments, the method further comprises predicting the subject to have functional loss of the STK11 tumor suppressor if the mean z-score is greater than 0.5. In some embodiments, the method further comprises administering to the subject a therapeutically effective amount of a treatment that is effective for subjects with mutations in the STK11 gene. In some embodiments, the therapeutic agent is an inhibitor of the Insulin-Like Growth Factor 1 Receptor. In some embodiments, the therapeutic agent is an inhibitor of a Cyclin-Dependent Kinase. In some embodiments, the therapeutic agent is an inhibitor of Cellular Inhibitor of Apoptosis 1 (cIAP1). In some embodiments, the Insulin-Like Growth Factor 1 Receptor inhibitor comprises linsitinib, or R1507. In some embodiments, the Cyclin-Dependent Kinase inhibitor comprises ribociclib, palbociclib, or abemeciclib. In some embodiments, the cIAP1 inhibitor comprises birinapant, xevinapant, tolianapant, or LC-161.

In some embodiments, the method further comprises predicting the subject to have functional loss of the STK11 tumor suppressor if the mean z-score is greater than 0.5. In some embodiments, the method further comprises administering to the subject a therapeutically effective amount of any treatment that has an FDA approved indication for treatment of subjects with mutations in the STK11 gene.

In some embodiments, the subject has one or more mutations in a STK11 gene. In some embodiments, the subject has a normal, or Wild-Type STK11 gene, for example, based on DNA sequencing of a tumor sample derived from the subject. In some embodiments, the subject's cancer has not been subjected to DNA sequencing, or the DNA sequencing is inconclusive, and the mutational status of the STK11 gene is unknown.

In some aspects, disclosed herein is a method of treating a cancer in a subject in need, comprising obtaining a biological sample from the subject, determining a level of one or more biomarkers in the biological sample, wherein the one or more biomarkers are one or more RNAs (e.g., mRNAs), or an expression product thereof, of the genes selected from the group of genes representing the STK11 Functional Loss Classifier (including, for example, AVPI1, BAG1, CPS1, DUSP4, FGA, GLCE, HAL, IRS2, MUC5AC, PDE4D, PTP4A1, RFK, SIK1, TACC2, TESC, and TFF1), determining an expression score for each of the one or more biomarkers in the biological sample relative to a reference control, determining a STK11 Functional Loss Score by integrating the one or more expression scores of the one or more biomarkers, and administering to the subject a therapeutically effective amount of an immunotherapeutic agent if the STK11 Functional Loss Score is lower than a predetermined threshold.

Accordingly, in some aspects, disclosed herein is a method of treating a cancer in a subject in need, comprising obtaining a biological sample from the subject, determining a level of one or more biomarkers in the biological sample, wherein the one or more biomarkers are one or more RNAs (e.g., mRNAs), or an expression product thereof, of the genes selected from the group of genes representing the STK11 Functional Loss Classifier (including, for example, AVPI1, BAG1, CPS1, DUSP4, FGA, GLCE, HAL, IRS2, MUC5AC, PDE4D, PTP4A1, RFK, SIK1, TACC2, TESC, and TFF1), determining a z-score or a singscore for each of the one or more biomarkers in the biological sample relative to a reference control, determining a z-score or a singscore by integrating the one or more z-scores or the one or more singscores of the one or more biomarkers, and administering to the subject a therapeutically effective amount of an immunotherapeutic agent if the z-score or the singscore is lower than 0.5.

In some aspects, disclosed herein is a method of treating a cancer in a subject in need, comprising obtaining a biological sample from the subject, determining a level of a first group of one or more biomarkers, the second group of one or more biomarkers, and the third group of one or more biomarkers in the biological sample, wherein the first group of one or more biomarkers comprise one or more RNAs (e.g., mRNAs), or an expression product thereof, of the genes selected from the group of genes representing the STK11 Functional Loss Classifier (including, for example, AVPI1, BAG1, CPS1, DUSP4, FGA, GLCE, HAL, IRS2, MUC5AC, PDE4D, PTP4A1, RFK, SIK1, TACC2, TESC, and TFF1); the second group of one or more biomarkers comprise one or more RNAs (e.g., mRNAs), or expression products thereof, of the genes selected from the group of genes representing the Neuroendocrine Differentiation Classifier (including, for example, ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4); and the third group of one or more biomarkers comprise one or more RNAs (e.g., mRNAs), or an expression product thereof, of the genes selected from the group of genes representing the TTF1 differentiation Classifier (including, for example, C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2), determining an expression score for each of the one or more biomarkers in the biological sample relative to a reference control, determining a first STK11 Functional Loss Score by integrating the one or more expression scores of the one or more biomarkers in the first group, a second Neuroendocrine Differentiation Score by using the one or more expression scores of the one or more biomarkers in the second group, and a third TTF1 Differentiation Score by using the one or more expression scores of the one or more biomarkers in the third group, and administering to the subject a therapeutically effective amount of a proinflammatory inhibitor if the first score (STK11 Functional Loss Score) is higher than a first threshold, the second score (Neuroendocrine Differentiation Score) is lower than a second threshold, and the third score (TTF1 Differentiation Score) is lower than a third threshold.

Accordingly, in some aspect, disclosed herein is a method of treating a cancer in a subject in need, comprising obtaining a biological sample from the subject, determining a level of a first group of one or more biomarkers, the second group of one or more biomarkers, and the third group of one or more biomarkers in the biological sample, wherein the first group of one or more biomarkers are one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers are one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers are one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining a z-score or a singscore for each of the one or more biomarkers in the biological sample relative to a reference control, determining a first z-score or a first singscore by integrating the z-scores of the one or more biomarkers in the first group, a second z-score or second singscore by integrating the z-scores of the one or more biomarkers in the second group, and a third z-score or a third singscore by integrating the z-scores of the one or more biomarkers in the third group, and administering to the subject a therapeutically effective amount of a proinflammatory inhibitor if the first z-score is higher than 0.5, the second z-score is lower than 0.5, and the third z-score is lower than 0.

In some embodiments, the subject has an increased level of one or more inflammatory biomarkers. In some embodiments, the one or more inflammatory biomarkers comprise natural killer (NK) cells, CD8+ T cells, polymorphonuclear myeloid-derived suppressor cells (PMN-MDSCs), IL-6, IL-15, IL-18, or IL-1.

In some embodiments, the proinflammatory inhibitor comprises lenvatinib, sitravatinib, bemcentinib, or an all-trans retinoic acid. In some embodiments, the proinflammatory inhibitor comprises a proinflammatory cytokine inhibitor. In some embodiments, the proinflammatory cytokine inhibitor is an inhibitor of IL-6, IL-15, IL-18, or IL-1.

In some aspects, disclosed herein is a method of treating a cancer in a subject in need, comprising obtaining a biological sample from the subject, determining a level of a first group of one or more biomarkers, the second group of one or more biomarkers, and the third group of one or more biomarkers in the biological sample, wherein the first group of one or more biomarkers comprise one or more RNAs (e.g., mRNAs), or an expression product thereof, of the genes selected from the group of genes representing the STK11 Functional Loss Classifier (including, for example, AVPI1, BAG1, CPS1, DUSP4, FGA, GLCE, HAL, IRS2, MUC5AC, PDE4D, PTP4A1, RFK, SIK1, TACC2, TESC, and TFF1); the second group of one or more biomarkers comprise one or more RNAs (e.g., mRNAs), or an expression product thereof, of the genes selected from the group of genes representing the Neuroendocrine Differentiation Classifier (including, for example, ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4); and the third group of one or more biomarkers comprise one or more RNAs (e.g., mRNAs), or an expression products thereof, of the genes selected from the group of genes representing the TTF1 differentiation Classifier (including, for example, C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2), determining an expression score for each of the one or more biomarkers in the biological sample relative to a reference control, determining a first STK11 Functional Loss Score by integrating the one or more expression scores of the one or more biomarkers in the first group, a second Neuroendocrine Differentiation Score by averaging the one or more expression scores of the one or more biomarkers in the second group, and a third TTF1 Differentiation Score by integrating the one or more expression scores of the one or more biomarkers in the third group, and administering to the subject a therapeutically effective amount of an immune checkpoint inhibitor if the first score (STK11 Functional Loss Score) is higher than a first threshold, the second score (Neuroendocrine Differentiation Score) is lower than a second threshold, and the third score (TTF1 Differentiation Score) is higher than a third threshold.

Accordingly, in some aspects, disclosed herein is a method of treating a cancer in a subject in need, comprising obtaining a biological sample from the subject, determining a level of a first group of one or more biomarkers, the second group of one or more biomarkers, and the third group of one or more biomarkers in the biological sample, wherein the first group of one or more biomarkers are one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers are one or more RNAs, or a an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers are one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining a z-score for each of the one or more biomarkers in the biological sample relative to a reference control, determining a first z-score by integrating the z-scores of the one or more biomarkers in the first group, a second z-score by averaging the z-scores of the one or more biomarkers in the second group, and a third z-score by averaging the z-scores of the one or more biomarkers in the third group, and administering to the subject a therapeutically effective amount of an immune checkpoint inhibitor if the first z-score is higher than 0.5, the second z-score is lower than 0.5, and the third z-score is higher than 0.

In some aspects, disclosed herein is a method of treating a cancer in a subject in need, comprising obtaining a biological sample from the subject, determining a level of a first group of one or more biomarkers and the second group of one or more biomarkers in the biological sample, wherein the first group of one or more biomarkers comprise one or more RNAs (e.g., mRNAs), or an expression product thereof, of the genes selected from the group of genes representing the STK11 Functional Loss Classifier (including, for example, AVPI1, BAG1, CPS1, DUSP4, FGA, GLCE, HAL, IRS2, MUC5AC, PDE4D, PTP4A1, RFK, SIK1, TACC2, TESC, and TFF1) and the second group of one or more biomarkers comprise one or more RNAs (e.g., mRNAs), or an expression product thereof, of the genes selected from the group of genes representing the Neuroendocrine Differentiation Classifier (including, for example, ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4), determining an expression score for each of the one or more biomarkers in the biological sample relative to a reference control, determining a first mean STK11 Functional Loss Score by averaging the one or more expression scores of the one or more biomarkers in the first group, a second mean Neuroendocrine Differentiation Score by integrating the one or more expression scores of the one or more biomarkers in the second group, and administering to the subject a therapeutically effective amount of a TRAF/cIAP inhibitor if the first score (STK11 Functional Loss Score) is higher than a first threshold and the second score (i.e., Neuroendocrine Differentiation Score) is higher than a second threshold.

Accordingly, in some aspects, disclosed herein is a method of treating a cancer in a subject in need, comprising obtaining a biological sample from the subject, determining a level of a first group of one or more biomarkers and the second group of one or more biomarkers, wherein the first group of one or more biomarkers are one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; and the second group of one or more biomarkers are one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4, determining a z-score or a singscore for each of the one or more biomarkers in the biological sample relative to a reference control, determining a first z-score or a first singscore by integrating the z-scores or the singscores of the one or more biomarkers in the first group, and a second z-score or a second singscore by integrating the z-scores or the singscores of the one or more biomarkers in the second group, and administering to the subject a therapeutically effective amount of a TRAF/cIAP inhibitor if the first z-score or the first singscore is higher than 0.5 and the second z-score or the second singscore is higher than 0.5.

In some embodiments, the subject has an anti-inflammatory tumor microenvironment. In some embodiments, the subject has a decreased expression level of one or more genes in the TNF/NFKB and/or JAK/STAT signaling pathways.

In some embodiments, the subject has an increased expression level of gene TRAF2.

In some embodiments, the TRAF/cIAP inhibitor is birinapant. In some embodiments, the TRAF/cIAP inhibitor is tolinapant. In some embodiments, the TRAF/cIAP inhibitor is xevinapant. In some embodiments, the TRAF/cIAP inhibitor can be used in combination with therapeutic radiotherapy treatments, or in combination with an immunotherapy agent (e.g., anti-PD1, anti-PDL1, or anti-CTLA4). In some embodiments, the TRAF/cIAP inhibitor can be any therapeutic agent inhibiting the effects of a cIAP1, cIAP2, or XIAP protein.

In some embodiments, the subject has one or more mutations in the STK11 gene. In some embodiments, the subject's cancer has a normal, or Wild-Type STK11 gene (e.g., based on DNA sequencing of a tumor tissue derived from the subject). In some embodiments, the subject's cancer has not been subjected to DNA sequencing, or the DNA sequencing is inconclusive, and the mutational status of the STK11 gene is unknown.

In some aspects, disclosed herein is a method of treating a cancer in a subject in need, comprising obtaining a biological sample from the subject, determining a level of a first group of one or more biomarkers, and the second group of one or more biomarkers, in the biological sample, wherein the first group of one or more biomarkers, the STK11 Functional Loss Classifier, is comprised of one or more mRNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers, the Neuroendocrine Differentiation Classifier, is comprised of one or more mRNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4, and the third group of one or more biomarkers, the TTF1 Differentiation Classifier, is composed of one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining an expression score for each of the one or more biomarkers in the biological sample relative to a reference control, determining a first STK11 Functional Loss Score by averaging the one or more expression scores of the one or more biomarkers in the first group, a second Neuroendocrine Differentiation Score by averaging the one or more expression scores of the one or more biomarkers in the second group, and a third TTF1 Differentiation Score by integrating the one or more expression scores of the one or more biomarkers in the third group, and administering to the subject a therapeutically effective amount of a proinflammatory inhibitor if the first score (i.e., STK11 Functional Loss Score) is higher than 0.5, the second score (i.e., Neuroendocrine Differentiation Score) is lower than 0.5, and the third score (i.e., TTF1 Differentiation Score) is lower than 0, administering to the subject a therapeutically effective amount of a checkpoint inhibitor if the first score (i.e., STK11 Functional Loss Score) is higher than 0.5, the second score (i.e., Neuroendocrine Differentiation Score) is lower than 0.5, and the third score (i.e., TTF1 Differentiation Score) is higher than 0, or administering to the subject a therapeutically effective amount of an inhibitor of TRAF/cIAP (i.e., Tolinapant, Xevinapant, Birinapant, LC-161) if the first score (STK11 Functional Loss Score) is higher than 0.5 and the second score (i.e., Neuroendocrine Differentiation Score) is higher than 0.5.

According, in some aspect, disclosed herein is a method of treating a cancer in a subject in need, comprising obtaining a biological sample from the subject, determining a level of a first group of one or more biomarkers, the second group of one or more biomarkers, and the third group of one or more biomarkers in the biological sample, wherein the first group of one or more biomarkers are one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers are one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers are one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2, determining a z-score or a singscore for each of the one or more biomarkers in the biological sample relative to a reference control, determining a first z-score or a first singscore by integrating the z-scores or the singscores of the one or more biomarkers in the first group, a second z-score by integrating the z-scores or the singscores of the one or more biomarkers in the second group, and a third z-score by integrating the z-scores or the singscores of the one or more biomarkers in the third group, and administering to the subject a therapeutically effective amount of a proinflammatory inhibitor if the first z-score or the first singscore is higher than 0.5, the second z-score or the second singscore is lower than 0.5, and the third z-score or the third singscore is lower than 0, administering to the subject a therapeutically effective amount of a checkpoint inhibitor if the first z-score or the first singscore is higher than 0.5, the second z-score or the second singscore is lower than 0.5, and the third z-score or the third singscore is higher than 0, or administering to the subject a therapeutically effective amount of an inhibitor of TRAF/cIAP if the first z-score or the first singscore is higher than 0.5 and the second z-score or the second singscore is higher than 0.5.

In some embodiments, the method of any preceding aspect further comprises determining a level of a fourth group of one or more biomarkers, wherein the fourth group of one or more biomarkers comprise one or more mRNAs, or an expression product thereof, of the genes selected from the group of genes representing the Gut-Like Differentiation Classifier (including, for example, HNF4A, MUC13, ANXA13, CDH17, CDX2, AKR1C4, ANKS4B, C12orf27, HNF1A, C10orf108, CDHR2, EPS8L3, TM4SF5, CDHR5, USH1C, HNF4A, SLC39A5, GPR35, MUC13, ANXA10, ANXA13, and CDH17, determining a z-score, a singscore, or similar relative expression score, for each of the one or more biomarkers in the biological sample relative to a reference control, and determining a fourth z-score or a fourth singscore by integrating the z-scores or the singscores of the one or more biomarkers in the fourth group.

In some embodiments, the method of any preceding aspect comprises determining a level of a fourth group of one or more biomarkers, wherein the fourth group of one or more biomarkers comprise one or more mRNAs, or an expression product thereof, of the genes selected from the group of genes representing the Squamous Differentiation Classifier (including, for example, GJB3, GJB5, KRT6A, KRT6B, KRT16, TRIM29, GJB3, GJB4, GJB5, ANXA8L2, ANXA8, KRT6A, KRT6C, KRT6B, KRT16, KRT17, TNS4, PLCD3, and LAMC2), determining a z-score, a singscore, or similar relative expression score, for each of the one or more biomarkers in the biological sample relative to a reference control, and determining a fourth z-score or a fourth singscore by integrating the z-scores or the singscores of the one or more biomarkers in the fourth group.

In some embodiments, the method of any preceding aspect further comprise determining a level of a fifth group of one or more biomarkers, wherein the fifth group of one or more biomarkers comprise one or more mRNAs, or an expression product thereof, of the genes selected from the group of genes representing the Squamous Differentiation Classifier (including, for example, GJB3, GJB5, KRT6A, KRT6B, KRT16, TRIM29, GJB3, GJB4, GJB5, ANXA8L2, ANXA8, KRT6A, KRT6C, KRT6B, KRT16, KRT17, TNS4, PLCD3, and LAMC2), determining a z-score, a singscore, or similar relative expression score, for each of the one or more biomarkers in the biological sample relative to a reference control, and determining a fifth z-score or a fifth singscore by integrating the z-scores or the singscores of the one or more biomarkers in the fifth group.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Methods

STK11-MLDD applies a validated gene signature to assess functional STK11 loss, then uses a rule-based classifier based on additional gene signatures to assign patients to one of three groups according to differentiation lineage: “Neuroendocrine,” “TTF1-Low,” and TTF1-Positive.” A retrospective and descriptive analysis was conducted across eight transcriptomic datasets of 2118 non-squamous NSCLC with 314 STK11 mutations, including 628 patients randomized to docetaxel or atezolizumab in the OAK and POPLAR clinical trials. TME is assessed by xCell immune deconvolution, and patient outcomes assessed by Cox proportional hazards regression models.

Statistical Analyses. Two-sided student's T-tests were used to compare statistical differences in continuous variable distributions between groups of samples: for instance, gene expression differences between LKB1 wild-type and mutant tumors. For comparison of discrete variables such as the presence of somatic mutations, statistical significance was determined using Fisher's exact test. Statistical associations between continuous variables such as LKB1 mRNA expression were determined using linear regression. Visualization of gene expression patterns by hierarchical clustering was performed with Cluster 3.0 and Tree View software packages. For the four expression clusters defined by hierarchical clustering, cluster “scores” were calculated for a given set of samples by averaging the standardized values of the respective genes. The score for the 16-gene LKB1-loss classifier, equivalent to the FOX/CREB cluster, was used for prediction of LKB1 status in clinical and cell line samples.

### Example 2: Results

Identification of differentiation phenotypes is currently dependent on histopathological evaluation, which is an imperfect tool and not well suited for clinical biomarker development. STK11-MLDD offers a more standardized rule-based classification based on discrete gene expression phenotypes. Moreover, the differentiation subgroups of STK11-MLDD have significant prognostic and predictive effects based on outcomes in patient cohorts and application of this classifier on randomized clinical trial data.

In addition to predicting immune outcomes, the present disclosure shows that the two immune-resistant subgroups, TTF1-Low and Neuroendocrine, both have similar levels of overall immune exclusion, but are associated with distinct differences in immune signaling and immunosuppressive cells within the immune microenvironment. Thus, STK11-MLDD can be used as a clinical biomarker for patient selection for next generation precision immune oncology approaches, enabling biomarker-based treatment strategies to be developed based on these unique differences in biology and STK11-MLDD serving to match patients with optimal treatments.

STK11-MLDD significantly outperforms STK11 mutations in predicting resistance to immunotherapy.
1. STK11-MLDD detects cases of functional STK11-Loss among putatively WT tumors that are associated with immunotherapy resistance.
2. STK11-MLDD of STK11-Deficient tumors with WT-Like biology that show improved response to immunotherapy.
3. STK11-MLDD identifies TTF1-Low and Neuroendocrine groups that are resistant to immunotherapy, while WT-Like and TTF1-Positive subgroups are immune responsive.
4. TTF1-Low has poor overall prognosis.
5. Functional STK11 loss can occur in the absence of detectable STK11 mutations, e.g. via whole exome sequencing. STK11-MLDD accurately detects cases of functional STK11 loss among the putatively WT tumors. Claim 1B: Conversely, among patients with STK11 mutations, STK11-MLDD classification of “WT-like” signature is associated with increased immune infiltration and improved response to Atezolizumab in OAK/POPLAR. Claim 1C: Both the Neuroendocrine and TTF1-Low groups have equivalently poor response to docetaxel and atezolizumab, while WT-Like and TTF1-Positive shows significantly better overall survival with atezolizumab over docetaxel (HR 0.5, P=0.03, Cox Proportional Hazards Model). Claim 1D: The TTF1-Low group has poor overall prognosis in OAK/POPLAR (HR 2.4, P=5.8e-10) as well as cohorts of resected lung cancer (P<3.8e-8).
   STK11-MLDD Subgroups Exhibit Distinctive Differences in TME Phenotypes that can be Used to Guide Precision Treatment Strategies.
1. TTF1-Low patients exhibit a chronically inflamed TME; STK11-MLDD shows utility in selecting patients to receive treatments targeting MDSCs (e.g., Lenvatinib, sitravatinib, bemcentinib, all-trans retinoic acid, inhibitors of S100A8/S100A9 function including inhibitory monoclonal antibodies, tasquinimod, or other inhibitors that act on this target). TTF-Low group is unique among the STK11-MLDD groups in showing increased markers of chronic inflammation including higher PMN-MDSC, CD8 and NK signatures, and pro-inflammatory cytokines IL1A, IL6, IL15, IL18. These differences persist after controlling for KRAS, KEAP1, and TP53 mutations. Claim 2B: Neuroendocrine tumors exhibit an anti-inflammatory TME; STK11-MLDD has utility in selecting patients to receive treatments targeting TRAF/cIAP pathway (e.g., Birinapant, Xevinapant, Tolinapant). In some embodiments, the TRAF/cIAP inhibitor can be used in in combination with therapeutic radiotherapy treatments, or in combination with an immunotherapy agent (e.g., anti-PD1, anti-PDL1, or anti-CTLA4). Neuroendocrine tumors have TME characterized by particularly low expression of genes in the TNF/NFKB and JAK/STAT signaling pathways (P<1e-10). It is shown that these genes are inversely associated with TRAF/cIAP signaling, specifically with high expression of TRAF2 (P<1e-6)—a known immune resistance protein-among STK11-Neuroendocrine NSCLC. Further, specifically among STK11-Neuroendocrine tumors a strong inverse association is observed between TRAF2 and immune infiltration (P<1e-6). In pre-clinical models, it is shown that inhibition of TRAF/cIAP using birinapant increases NFKB signaling and synergistically enhances anti-tumor immune response against STK11-deficient NSCLC cell lines.

### CONCLUSIONS

STK11-MLDD is highly predictive of immune exclusion and NSCLC patient outcomes. It identifies STK11-deficient subsets with increased immune infiltration and improved ICB response, and two putatively immune-resistant subgroups with markedly different TME phenotypes: Neuroendocrine tumors have an anti-inflammatory phenotype, while TTF1-Low tumors exhibit chronic inflammation.

STK11 Functional Loss Classifier (n=16): AVPI1, PAG1, DUSP4, GLCE, IRS2, PDE4D, PTP4A1, RFK, SIK1, TACC2, TESC, CPS1, FGA, HAL, MUC5AC

TTF1 Differentiation Classifier (n=23): C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2

Neuroendocrine Differentiation Classifier (n=26), AGXT2L1, COL25A1, SLC14A2, ODC1, ENO3, ASCL1, CALCA, CALCB, RET, ZMAT4, MTMR7, SLC38A8, LOC100190940, CELF3, C1orf95, KLK11, KLK13, KLK12, KLK14, SEC11C, BAALC, C8orf56, LOC145837, ERO1LB, CTNND2, UGT3A1

Gut-Like Differentiation Classifier (n=17), AKR1C4, ANKS4B, C12orf27, HNF1A, c10orf108, CDHR2, EPS8L3, TM4SF5, CDHR5, USH1C, HNF4A, SLC39A5, GPR35, MUC13, ANXA10, ANXA13, CDH17

Squamous Differentiation Classifier, (n=14), TRIM29, GJB3, GJB4, GJB5, ANXA8L2, ANXA8, KRT6A, KRT6C, KRT6B, KRT16, KRT17, TNS4, PLCD3, LAMC2 Pathway Dependence Classifier (n=9), IGF1R, FURIN, INSL4, KLF5, HNF1B, FOXA1, FOXA2, FOXA3, FOXK1

The biomarkers disclosed herein can be used for the following treatment strategies:
1. Treatment strategies that target IGF1R
2. Treatment strategies that target cyclin-dependent kinases (such as Palbociclib, ribociclib, abemeciclib
3. Treatment strategies that target developmental pathways specific to the differentiation state of the tumor, such as inhibitors of the Wnt pathway

The steps include:
1. Signature Scores:
   Primary scores:
      1) STK11 Loss Signature
      2) Neuroendocrine Signature
      3 TTF1 Signature
   Secondary scores:
      4) Gut Signature
      5) Squamous Signature
      6) Pathway Dependence Signature

The use of the Gut, Squamous, and Pathway Dependence signatures can be used in conjunction with the main primary scores to define tumors with altered pathway activity that can be used to select targeted treatments.
1) Gut, Squamous, and Pathway Dependence Signatures can be linked to evidence of dysregulated developmental pathways and can be used to identify tumors with potential susceptibility to WNT (e.g. tegavivant), NOTCH (including gamma secretase inhibitors such as Nirogacestat), or Hedgehog targeted agents (sonidegib, vismodegib)
2) Pathway Dependence Signature genes IGF1R, IRS2, FURIN, INSL4 can be used to select patients to receive IGF1R inhibitors including monoclonal antibodies (e.g. R1507) and small molecule inhibitors (e.g. linsitinib).
3) Pathway Dependence Signature genes IGF1R, IRS2, FURIN, INSL4 can be used to select patients to receive cyclin-dependent kinase inhibitors (e.g. ribociclib, palbociclib, abemeciclib)
4) Pathway Dependence Signature genes IGF1R, IRS2, FURIN, INSL4 can be used to select patients to receive cIAP1 targeted agents (e.g. birinapant, xevinapant, tolianapant, LC-161)
5) STK11 Loss Signature can be used to select patients to receive IGF1R inhibitors including monoclonal antibodies (e.g. R1507) and small molecule inhibitors (e.g. linsitinib).
6 STK11 Loss Signature can be used to select patients to receive cyclin-dependent kinase inhibitors (e.g. ribociclib, palbociclib, abemeciclib)
7) STK11 Loss Signature can be used to select patients to receive cIAP1 targeted agents (e.g. birinapant, xevinapant, tolianapant, LC-161)

All of these embodiments above can state that a score cutoff >0.5 would be used to identify patients who would be susceptible to these agents.

### Example 3: Classifying a Tumor According to STK11-Loss and Multilineage De-Differentiation: ‘STK11-MLDD’ Using Biomarker Scores from Three Biomarker Gene Sets

Herein, three primary gene sets are described for 1) STK11 Functional Loss Signature, 2) Neuroendocrine Signature, 3) TTF1 Signature. Scores are calculated according to the above method, using either the ‘Mean Z-score’ method or the ‘Singscore’ method, and yielding the binary classifier determination for each signature. To determine the STK11-MLDD for a given patient sample, these scores are applied in a stepwise approach—
1) First by applying the STK11 Functional Loss Signature
2) Then applying the Neuroendocrine Signature to the STK11-Loss tumors
3) Then applying the TTF1 Signature to the non-neuroendocrine tumors.
   This results in four classifications: 1) STK11 WT-like, 2) STK11 Loss Neuro, 3) STK11 Loss TTF1-Low, and 4) STK11 Loss TTF1-Positive.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Gene Sets

| Primary | Primary | Primary | Additional | Additional | Additional |
|---|---|---|---|---|---|
| gene sets | gene sets | gene sets | gene sets | gene sets | gene sets |
| STK11LossSignature | Neuroendocrine | TTF1 | Gut-like | Squamous | Pathway |
| | | | | | dependence |
| AVPI1 | AGXT2L1 | C1orf116 | AKR1C4 | TRIM29 | IGF1R |
| BAG1 | ASCL1 | CISH | ANKS4B | GJB3 | FURIN |
| CPS1 | BAALC | FAM184A | C12orf27 | GJB4 | INSL4 |
| DUSP4 | C1orf95 | FBP1 | HNF1A | GJB5 | KLF5 |
| FGA | C8orf56 | HOPX | C10orf108 | ANXA8L2 | FGL1 |
| GLCE | CALCA | IRX5 | CDHR2 | ANXA8 | HNF1B |
| HAL | CALCB | KCNK5 | EPS8L3 | KRT6A | BCAS1 |
| IRS2 | CELF3 | LMO3 | TM4SF5 | KRT6C | CASP9 |
| MUC5AC | COL25A1 | LPCAT1 | CDHR5 | KRT6B | FOXA1 |
| PDE4D | CTNND2 | MBIP | USH1C | KRT16 | |
| PTP4A1 | ENO3 | NKX2-1 | HNF4A | KRT17 | |
| RFK | ERO1LB | PLCH1 | SLC39A5 | TNS4 | |
| SIK1 | KLK11 | PPP1R13B | GPR35 | PLCD3 | |
| TACC2 | KLK12 | RPS6KA2 | MUC13 | LAMC2 | |
| TESC | KLK13 | SCNN1B | ANXA10 | | |
| TFF1 | KLK14 | SELENBP1 | ANXA13 | | |
| | LOC100190940 | SFTPB | CDH17 | | |
| | LOC145837 | SLC34A2 | | | |
| | MTMR7 | ST3GAL5 | | | |
| | ODC1 | STXBP1 | | | |
| | RET | TMPRSS2 | | | |
| | SEC11C | | | | |
| | SLC14A2 | | | | |
| | SLC38A8 | | | | |
| | UGT3A1 | | | | |
| | ZMAT4 | | | | |

TABLE 2
Developmental Pathway Dysregulation
associated with GutSignature

| Drug or Genetic | | GutSignature Other |
|---|---|---|
| Dependency | Gut Signature NSCLC | Cancer Types |
| CRISPR.CTNNB1 | NS | *** |
| CRISPR.FZD5 | * | *** |
| CRISPR.KLF5 | ** | *** |
| CRISPR.LGR4 | * | |
| RNAi.CTNNB1 | *** | *** |
| RNAi.FZD5 | ** | |
| FLI.06 (NOTCHi) | * | |
| Sonidegib (HEDGEHOGi) | ** | |
| NVP.ADW742 | ** | * |
| WNT11 | *** | * |
| WNT5B | * | *** |
| WNT9B | *** | ** |
| FZD5 | *** | *** |
| AXIN2 | * | *** |
| LGR4 | * | *** |

* P < 0.05;
** P < 0.01;
*** P < 0.001

TABLE 3
Gene Signatures

| | Number of | |
|---|---|---|
| Gene Set Name | Genes | Genes in Signature |
| STK11 | 16 | AVP1, BAG1, DUSP4, GLCE, IRS2, |
| Functional | | PDE4D, PTP4A1, RFK, SIK1, TACC2, |
| Loss | | TESC, TFF1, CPS1, FGA, HAL, |
| Signature | | MUC5AC |
| Neuroendocrine | 22 | AGXT2L1, COL25A1, SLC14A2, ODC1, |
| Signature | | ENO3, ASCL1, CALCA, CALCB, RET, |
| | | ZMAT4, MTMR7, SLC38A8, |
| | | LOC100190940, CELF3, C1orf95, |
| | | KLK11, KLK13, KLK12, KLK14, |
| | | SEC11C, BAALC, C8orf56, LOC145837, |
| | | ERO1LB, CTNND2, UGT3A1 |
| TTF1 Signature | 22 | C1orf116, CISH, FAM184A, FBP1, |
| | | HOPX, IRX5, KCNK5, LMO3, LPCAT1, |
| | | MBIP, NKX2-1, PLCH1, PPP1R13B, |
| | | RPS6KA2, SCNN1B, SELENBP1, |
| | | SFTPB, SLC34A2, ST3GAL5, STXBP1, |
| | | TMPRSS2, WFDC2 |

TABLE 4
Gene Set Score Cutoffs

| | Mean Z. Score | Singscore |
|---|---|---|
| Gene Set | Cutoff* | Cutoff* |
| STK11 Functional Loss | 0.42 | 0.1 |
| Signature | | |
| Neuroendocrine Signature | 0.5 | −0.2 |
| TTF1 Signature | 0 | 0.2 |

What is claimed is:

1. A method of predicting responsiveness of a subject having a cancer to an immunotherapeutic agent, comprising
obtaining a biological sample from the subject;
determining a level of one or more biomarkers in the biological sample, wherein the one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1;
determining an expression score for each of the one or more biomarkers in the biological sample relative to a reference control;
determining a mean expression score by averaging the one or more expression scores of the one or more biomarkers; and
predicting the subject as responsive to the immunotherapeutic agent if the mean expression score is lower than a threshold.

2. The method of claim 1, wherein the expression score is a z-score and the mean expression score is a mean z-score by averaging the one or more z-scores of the one or more biomarkers.

3. The method of claim 2, comprising predicting the subject as responsive to the immunotherapeutic agent if the mean z-score is lower than 0.5.

4. The method of any one of claims 1-3, further comprising administering to the subject a therapeutically effective amount of the immunotherapeutic agent.

5. The method of any one of claims 1-4, wherein the immunotherapeutic agent is an immune checkpoint inhibitor.

6. The method of claim 5, wherein the checkpoint inhibitor comprises a PD-1 inhibitor, a PD-L1 inhibitor, a PD-L2 inhibitor, or a CTLA-4 inhibitor.

7. The method of claim 6, wherein the checkpoint inhibitor comprises Pembrolizumab, Nivolumab, Cemiplimab, Atezolizumab, Durvalumab, Ipilimumab, or Tremeliumumab.

8. The method of any one of claims 1-7, wherein the subject has one or more mutations in a STK11 gene.

9. The method of any one of claims 1-7, wherein the subject has a normal STK11 gene.

10. A method of treating a cancer in a subject in need, comprising
obtaining a biological sample from the subject;
determining a level of one or more biomarkers in the biological sample, wherein the one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1;
determining an expression score for each of the one or more biomarkers in the biological sample relative to a reference control;
determining a mean expression score by averaging the one or more expression scores of the one or more biomarkers; and
administering to the subject a therapeutically effective amount of an immunotherapeutic agent if the mean expression score is lower than a threshold.

11. A method of treating a cancer in a subject in need, comprising
obtaining a biological sample from the subject;
determining a level of a first group of one or more biomarkers, a second group of one or more biomarkers, and a third group of one or more biomarkers in the biological sample, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers comprise one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2,
determining an expression score for each of the one or more biomarkers in the biological sample relative to a reference control;
determining a first expression score by integrating the expression scores of the one or more biomarkers in the first group, a second expression score by integrating the expression score of the one or more biomarkers in the second group, and a third expression score by integrating the expression score of the one or more biomarkers in the third group; and
administering to the subject a therapeutically effective amount of a proinflammatory inhibitor if the first expression score is higher than a first threshold, the second expression score is lower than a second threshold, and the third expression score is lower than a third threshold.

12. The method of claim 11, comprising administering to the subject a therapeutically effective amount of a proinflammatory inhibitor if the first expression score is higher than 0.5, the second expression score is lower than 0.5, and the third expression score is lower than 0.

13. The method of claim 11 or 12, wherein the subject has an increased level of one or more inflammatory biomarkers.

14. The method of claim 13, wherein the one or more inflammatory biomarkers comprise natural killer (NK) cells, CD8+ T cells, polymorphonuclear myeloid-derived suppressor cells (PMN-MDSCs), IL-6, IL-15, IL-18, or IL-1.

15. The method of any one of claims 11-14, wherein the proinflammatory inhibitor comprises lenvatinib, sitravatinib, bemcentinib, or an all-trans retinoic acid.

16. The method of any one of claims 11-14, wherein the proinflammatory inhibitor comprises a proinflammatory cytokine inhibitor.

17. The method of claim 16, wherein the proinflammatory cytokine inhibitor is an inhibitor of IL-6, IL-15, IL-18, or IL-1.

18. The method of any one of claims 11-17, wherein the subject has one or more mutations in a STK11 gene.

19. The method of any one of claims 11-17, wherein the subject has a normal STK11 gene.

20. A method of treating cancer in a subject in need, comprising
obtaining a biological sample from the subject;
determining a level of a first group of one or more biomarkers, a second group of one or more biomarkers, and a third group of one or more biomarkers in the biological sample, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers comprise one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2,
determining an expression score for each of the one or more biomarkers in the biological sample relative to a reference control;
determining a first expression score by integrating the expression scores of the one or more biomarkers in the first group, a second expression score by integrating the expression score of the one or more biomarkers in the second group, and a third expression score by integrating the expression score of the one or more biomarkers in the third group; and
administering to the subject a therapeutically effective amount of a proinflammatory inhibitor if the first expression score is higher than a first threshold, the second expression score is lower than a second threshold, and the third expression score is higher than a third threshold.

21. The method of claim 20, comprising administering to the subject a therapeutically effective amount of an immune checkpoint inhibitor if the first z-score is higher than 0.5, the second z-score is lower than 0.5, and the third z-score is higher than 0.

22. The method of claim 21, wherein the checkpoint inhibitor comprises a PD-1 inhibitor, a PD-L1 inhibitor, a PD-L2 inhibitor, or a CTLA-4 inhibitor.

23. The method of claim 21, wherein the checkpoint inhibitor comprises Pembrolizumab, Nivolumab, Cemiplimab, Atezolizumab, Durvalumab, Ipilimumab, or Tremeliumumab.

24. The method of any one of claims 20-23, wherein the subject has one or more mutations in a STK11 gene.

25. The method of any one of claims 20-23, wherein the subject has a normal STK11 gene.

26. A method of treating a cancer in a subject in need, comprising
obtaining a biological sample from the subject;
determining a level of a first group of one or more biomarkers and a second group of one or more biomarkers, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1 and the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4;
determining an expression score for each of the one or more biomarkers in the biological sample relative to a reference control;
determining a first expression score by integrating the expression scores of the one or more biomarkers in the first group and a second expression score by integrating the expression score of the one or more biomarkers in the second group; and
administering to the subject a therapeutically effective amount of a TRAF/cIAP inhibitor if the expression score is higher than a first threshold and the second expression score is higher than a second threshold.

27. The method of claim 26, comprising administering to the subject a therapeutically effective amount of a TRAF/cIAP inhibitor if the first expression score is higher than 0.5 and the second expression is higher than 0.5.

28. The method of any one of claims 26-27, wherein the subject has an anti-inflammatory tumor microenvironment.

29. The method of any one of claims 26-28, wherein the subject has a decreased expression level of one or more genes in the TNF/NFKB and/or JAK/STAT signaling pathways.

30. The method of any one of claims 26-29, wherein the subject has an increased expression level of gene TRAF2.

31. The method of any one of claims 26-30, wherein the TRAF/cIAP inhibitor comprises Birinapant, Tolinapant, Xevinapant, or LC-161.

32. The method of any one of claims 26-31, wherein the subject has one or more mutations in a STK11 gene.

33. The method of any one of claims 26-31, wherein the subject has a normal STK11 gene.

34. A method of treating a cancer in a subject in need, comprising
obtaining a biological sample from the subject;
determining a level of a first group of one or more biomarkers, a second group of one or more biomarkers, and a third group of one or more biomarkers in the biological sample, wherein the first group of one or more biomarkers comprise one or more RNAs, or an expression product thereof, of the genes selected from the group consisting of AVPI1, BAG1, CPS1, DUSP4, FGA, GLCE, HAL, IRS2, MUC5AC, PDE4D, PTP4A1, RFK, SIK1, TACC2, TESC, and TFF1; the second group of one or more biomarkers comprise one or more RNAs, or an expression product thereof, of the genes selected from the group consisting of ASCL1, CALCA, CALCB, RET, KLK12, AGXT2L1, BAALC, C1orf95, C8orf56, CELF3, COL25A1, CTNND2, ENO3, ERO1LB, KLK11, KLK13, KLK14, LOC100190940, LOC145837, MTMR7, ODC1, SEC11C, SLC14A2, SLC38A8, UGT3A1, and ZMAT4; and the third group of one or more biomarkers comprise one or more RNAs, or an expression product thereof, of the genes selected from the group consisting of C1orf116, CISH, FAM184A, FBP1, HOPX, IRX5, KCNK5, LMO3, LPCAT1, MBIP, NKX2-1, PLCH1, PPP1R13B, RPS6KA2, SCNN1B, SELENBP1, SFTPB, SLC34A2, ST3GAL5, STXBP1, TMPRSS2, and WFDC2,
determining a z-score, or similar relative expression score, for each of the one or more biomarkers in the biological sample relative to a reference control;
determining a first z-score by integrating the z-scores of the one or more biomarkers in the first group, a second z-score by integrating the z-scores of the one or more biomarkers in the second group, and a third z-score by integrating the z-scores of the one or more biomarkers in the third group; and
administering to the subject a therapeutically effective amount of a proinflammatory inhibitor if the first z-score is higher than 0.5, the second z-score is lower than 0.5, and the third z-score is lower than 0;
administering to the subject a therapeutically effective amount of a checkpoint inhibitor if the first z-score is higher than 0.5, the second z-score is lower than 0.5, and the third z-score is higher than 0; or
administering to the subject a therapeutically effective amount of an inhibitor of TRAF/cIAP if the first z-score is higher than 0.5 and the second z-score is higher than 0.5.

35. The method of any one of claims 1-34, further comprising
determining a level of a fourth group of one or more biomarkers, wherein the fourth group of one or more biomarkers comprise one or more RNAs, or expression products thereof, of the genes selected from the group consisting of HNF4A, MUC13, ANXA13, CDH17, CDX2, AKR1C4, ANKS4B, C12orf27, HNF1A, C10orf108, CDHR2, EPS8L3, TM4SF5, CDHR5, USH1C, HNF4A, SLC39A5, GPR35, MUC13, ANXA10, ANXA13, and CDH17;
determining a z-score, or similar relative expression score, for each of the one or more biomarkers in the biological sample relative to a reference control; and
determining a fourth z-score by integrating the z-scores of the one or more biomarkers in the fourth group.

36. The method of any one of claims 1-35, further comprising
determining a level of a fifth group of one or more biomarkers, wherein the fifth group of one or more biomarkers comprise one or more RNAs, or expression products thereof, of the genes selected from the group consisting of GJB3, GJB5, KRT6A, KRT6B, KRT16, TRIM29, GJB3, GJB4, GJB5, ANXA8L2, ANXA8, KRT6A, KRT6C, KRT6B, KRT16, KRT17, TNS4, PLCD3, and LAMC2;
determining a z-score, or similar relative expression score, for each of the one or more biomarkers in the biological sample relative to a reference control; and
determining a fifth z-score by integrating the z-scores of the one or more biomarkers in the fifth group.
